(19) United States
(12) Patent Application Publication
(10) Pub. No.: US 2011/0224407 A1
(43) Pub. Date: Sep. 15, 2011

(54) Type I Interferon Antagonists

(75) Inventors: Jerome Langer, Highland Park, NJ (US); Gideon Schreiber, Rehovot (IL)

(73) Assignees: UNIVERSITY OF MEDICINE AND DENTISTRY OF NEW JERSEY, Somerset, NJ (US); YEDA RESEARCH AND DEVELOPMENT CO., LTD., Rehovot (IL)

(21) Appl. No.: 13/063,108

(22) PCT Filed: Sep. 9, 2009
(86) PCT No.: PCT/US09/56366

§ 371 Date: May 25, 2011

Related U.S. Application Data
(60) Provisional application No. 61/191,507, filed on Sep. 9, 2008.

(51) Int. Cl. C07K 14/555

(52) U.S. Cl. 530/351

(57) ABSTRACT

Disclosed in certain embodiments is a method of preparing a Type 1 interferon antagonist comprising modifying a Type 1 interferon at the site of interaction with the interferon receptor subunit IFNAR-1 such that the binding affinity of the interferon to the IFNAR-1 subunit is reduced as compared to the native interferon, and corresponding compositions and methods of treatment thereof.

### CLAIM OF PRIORITY

This application claims priority to U.S. Provisional Application No. 61/191,507, filed Sep. 9, 2008 the disclosure of which is hereby incorporated by reference.

### FIELD OF THE INVENTION

This invention relates to Type I interferon antagonists, their preparation and their use.

### BACKGROUND

Type I interferons are a family of proteins that constitute a rapid and broad-spectrum defensive response to viral infections and some intracellular parasites. These proteins have therapeutic use against some viral diseases, several tumors, and multiple sclerosis. However, there is recent evidence that Type I interferons are inappropriately produced in certain disease states, and are involved in the cause or progression of autoimmune disease states such as lupus (systemic lupus erythematosus) and Sjögren's syndrome. In such instances, it has been suggested that pharmacologic blockade of interferon action might be an effective method of slowing or stopping the progression of the disease. There may also be other situations in humans where blocking Type I IFN action is desirable. In addition, there are a number of studies in animals, including mouse strains that are susceptible to autoimmune disease, where Type I interferon may be involved in promoting pathogenesis. Therefore, therapeutic approaches to blocking the action of native interferon are required both for humans and for experimental species such as mice.

Type I interferons, including human interferons alpha, beta, omega, kappa and epsilon are well studied cytokines whose main role appears to be rapid and broad-spectrum antiviral protection. Despite the high homology and sequence conservation of these different IFN subtypes, individual subtypes display different profiles of biological activities including antiproliferative, antiviral, and immunomodulatory. Type I IFNs also have a number of other functions, and affect many parts of the immune system. The Type I interferon family of proteins is classified together because of the relationship of the protein/gene structures and sequences, and because all of the proteins exert their action on cells by binding to a common cell-surface receptor. The receptor, IFNAR, is composed of two transmembrane protein subunits, IFNAR-1 and IFNAR-2. Data demonstrate that Type I interferons generally bind more tightly to IFNAR-2, and have relatively weak binding to IFNAR-1. This and other results have led to a model of IFN action wherein IFN binds with high affinity to IFNAR-2 to form a binary complex. This complex then recruits or re-aligns IFNAR-1 to form a ternary complex. Assembly of the ternary complex leads to intracellular signaling and the various biochemical and physiological effects of IFN.

### OBJECTS AND SUMMARY OF THE INVENTION

It is an object of the present invention to provide antagonists for Type I interferon and corresponding methods of synthesis and methods of treatment thereof.

In certain embodiments, the present invention is directed to a method of preparing a Type II interferon antagonist comprising modifying a Type I interferon at the site of interaction with the interferon receptor subunit IFNAR-1 such that the binding affinity of the interferon to the IFNAR-1 subunit is reduced as compared to the native interferon.

In certain embodiments, the present invention is directed to a method of preparing a Type I interferon antagonist comprising modifying a Type I interferon at the site of interaction with the interferon receptor subunit IFNAR-1 such that the binding affinity of the interferon to the IFNAR-1 subunit is reduced as compared to the native interferon such that the binding affinity of the interferon to the IFNAR-2 subunit is maintained as compared to the native interferon

In certain embodiments, the present invention is directed to a method of preparing a Type I interferon antagonist comprising modifying a Type I interferon at the site of interaction with the interferon receptor subunit IFNAR-1 such that the binding affinity of the interferon to the IFNAR-1 subunit is reduced as compared to the native interferon and further comprising modifying the interferon at the site of interaction with the interferon receptor subunit IFNAR-2 such that the binding affinity of the interferon to the IFNAR-2 subunit is increased as compared to the native interferon.

In certain embodiments, the present invention is directed to a method of preparing a Type I interferon antagonist comprising modifying an interferon such that (i) the binding affinity of the interferon to the IFNAR-1 subunit is reduced as compared to the native interferon and (ii) the binding affinity of the interferon to the IFNAR-2 subunit is increased as compared to the native interferon.

In the methods disclosed herein, the interferon can be selected from e.g., the group consisting of IFN-α, IFN-β, IFN-ω, IFN-κ, IFN-ε, IFN-τ, IFN-ζ/limitin, IFN-δ and IFN-ν. The interferon can originate from a mammal, e.g., a human or mouse.

In the methods disclosed herein, the modifying can comprise, e.g., mutating, one or more amino acids in the IFNAR-1 binding region of the interferon.

In the methods disclosed herein, the interferon can be IFN-α2, preferably IFN-α2a or IFN-α2b.

In embodiments directed to IFN-α2b, the interferon can be modified at one or more amino acid positions in region 120-125. For example, the interferon can be modified at one or more sites selected from the group consisting of Arg120, Lys121 and Gln124. In certain embodiments, the Arg 120 of the IFN-α2b is substituted with Glu, the Arg 120 of the IFN-α2b is substituted with Glu and/or the Lys 122 is substituted with Glu.

In certain embodiments, the present invention is directed to a Type I interferon produced according to any of the methods of disclosed herein.

In certain embodiments, the present invention is directed to a Type I interferon that has sufficiently low binding affinity to the interferon receptor subunit IFNAR-1 such that the interferon exhibits antagonist activity.

In certain embodiments, the present invention is directed to a Type I interferon that has (i) sufficiently low binding affinity to the interferon receptor subunit IFNAR-1 such that the interferon exhibits antagonist activity and (ii) sufficient binding affinity to the interferon receptor subunit IFNAR-2 to interfere with the binding of a native or endogenous interferon

The Type I interferons of the present invention can be selected from, e.g., the group consisting of IFN-α, IFN-β, IFN-ω, IFN-κ, IFN-ε, IFN-τ, IFN-ζ/limitin, IFN-δ and IFN-ν. The interferon can originate from a mammal, e.g., a human or mouse.

The Type I interferons of the present invention can be IFN-α2, preferably IFN-α2a or IFN-α2b. In embodiments directed to IFN-α2b, the interferon can be modified at one or more amino acid positions in region 120-125. For example, the interferon can be modified at one or more sites selected from the group consisting of Arg120, Lys121 and Gln124. In certain embodiments, the Arg 120 of the IFN-α2b is substituted with Glu, the Arg 120 of the IFN-α2b is substituted with Glu and/or the Lys 122 is substituted with Glu.

In certain embodiments, the present invention is directed to a method of antagonizing the effects of interferon comprising contacting an interferon receptor with a Type I interferon antagonist as disclosed herein. The contacting can be in-vitro or in-vivo.

In certain embodiments, the present invention is directed to a method of treating a disease or condition in a mammal comprising administering a Type I interferon antagonist as disclosed herein in an effective amount to antagonize the effects of a native or endogenous interferon.

In certain embodiments, the present invention is directed to a method of treating a disease or condition in a mammal comprising administering a nucleic acid encoding a Type I interferon antagonist as disclosed herein in an effective amount to antagonize the effects of a native or endogenous interferon. The nucleic acid can comprises DNA or RNA. In alternative embodiments, the nucleic acid is contained within a vector and can be, e.g., a plasmid or a virus.

In the methods disclosed herein, the disease or condition can be, e.g., auto-immune mediated. In certain embodiments, the disease or condition is selected from the group consisting of systemic lupus erythematosus, Sjogren's syndrome, Type 1 diabetes, polymyositis, and periodontitis. In alternative embodiments, the administration is associated with allogeneic grafts or transplants. Further, the administration can be selected from e.g., the group consisting of parenteral, intradermal, intramuscular, intraperitoneal, intravenous, subcutaneous, intranasal, epidural, topical, pulmonary and oral routes.

In certain embodiments, the present invention is directed to a pharmaceutical composition comprising an interferon as disclosed herein and a pharmaceutically acceptable excipient. The composition can be, e.g., in a form selected from the group consisting of a solution, suspension, emulsion, tablet, capsule, powder and sustained-release formulation.

In certain embodiments, the present invention is directed to a nucleic acid encoding a Type I interferon antagonist as disclosed herein. The nucleic acid can comprises DNA or RNA. In alternative embodiments, the nucleic acid is contained within a vector and can be, e.g., a plasmid or a virus.

References to specific human IFN-α amino acid positions are made throughout this disclosure. Numbering systems for such references vary among different published references but can be correlated and easily identified. The two common numbering systems are derived from the alignment of amino acids of the family of human IFN-αs, most of which have 166 amino acids, versus the sequence of human IFN-α2, which has only 165 amino acids. As an example, using the 166-amino acid sequence convention for human IFN-αs, the arginine amino acid of IFN-α2b that can be mutated to glutamate with consequent loss of normal biological activity is arginine 121. However, using the distinct numbering of the 165-amino acid IFN-α2b itself, this is arginine 120. These differences are known and understood by practitioners of the art, and both numbering conventions are used in this disclosure.

It is further noted that multiple DNA sequence triplets can encode a particular amino acid, making it possible to encode any specified sequence of amino acids (i.e. a protein) with more than one sequence of deoxyoligonucleotides. Accordingly, any DNA sequence that encodes an amino acid sequence for a Type I interferon antagonist constructed according to this disclosure shall be deemed to have also been disclosed by this application.

### BRIEF DESCRIPTION OF THE DRAWINGS

FIG. 1 is a schematic of the design of an IFN antagonist. (Left panel): IFN normally has sites 1 and 2 for interacting productively with the interferon receptor subunits IFNAR-1 and IFNAR-2, respectively, to form a ternary complex of IFN/IFNAR-1/IFNAR-2, which then generates an intracellular signal. (Right Panel): To form an antagonist, the site on IFN for interacting with IFNAR-1 (Site 1) is mutated (denoted by “X”). This antagonist can then form the binary IFN/IFNAR-2 complex, but cannot recruit IFNAR-1 into the complex. The antagonist competes with native IFNs for IFNAR-2 to block their action, but produces no signal and therefore no biological effects.

FIG. 2 depicts the ability of selected mutants to antagonize antiviral protection conferred by native IFN-α2. Row 1 shows the titration by two-fold serial dilution of the active IFN-α2 used to test the IFN-α2 mutants specified in Rows 2-8. In Rows 2-8, the indicated mutants are added with the highest concentration in the left-most well, and then serially diluted, as indicated through well 11. For each mutant, the right-most well (Well 12) contains the mutant at the highest concentration used to test antagonism (the same concentration as in the 1st well of each dilution series), but with no native IFN-α2. Thus, the final well in each series (“+”) indicates whether the mutant itself, at high concentration, has IFN antiviral activity. Shaded wells: cells protected by IFN. Unshaded wells: lack of IFN protection and cells killed.

FIG. 3 depicts the alignment of human and murine Type I interferon sequences for helices B, C and D. Experimentally determined α-helices are indicated in italics and underlined (PDB entries: HuIFN-α2, HTF, HuIFN-β, IAU1; MuIFN-β, HFA). Sequence numbers for the first residue of each segment are indicated for IFN-α2 and are the same for IFN-α2/α1. Other human IFN-αs contain 1 additional amino acid, generally indicated as residue #44 (which is missing in IFN-α2). Thus, papers using the “consensus” human IFN-α sequence numbering have all homologous positions above residue #43 one number higher than the specific sequence of HuIFN-α2 or its derivative IFN-α2/α1 (e.g., residue #120 in HuIFN-α2 is homologous to residue #121 in other human IFN-αs).

FIG. 4 depicts antiviral and antiproliferative activity of human IFN-α2 mutants. For each mutant indicated, activity is presented as a fraction of the native IFN-α2, measured on human WISH cells. Thus, on the logarithmic scale, “0” is the base-line wild-type activity, and mutants show decreases of up to 5 logs activity (this number represents no activity at the highest concentration of IFN mutant used in the assays; the mutants may have even less activity, as indicated by the designation of “≦” in Tables 1-3). Mutants were measured between 1 and 5 times each in independent assays. For multiple measurements, error bars indicate the Standard Error (SEM).

FIG. 5 depicts the comparison of antiviral antagonist potency for IFN-α2 mutants with antagonist activity. Relative potencies of antagonism of antiviral activity (measured on HeLa cells) are expressed as the ratio of IC₅₀ values for different IFN-α2 mutants relative to that of IFN-α2[R120E1]. Values derive from 1-3 independent measurements of each mutant, with an internal R120E mutant standard in each assay to correct for inter-assay variability of absolute IC₅₀ values. Mutants with values higher than 1.0 have higher potency than IFN-α2[R120E], and values lower than 1.0 have lower potency than R120E.

### DETAILED DESCRIPTION

This application relates to novel antagonists of Type I interferon that can be created by disrupting the site for interaction with the interferon receptor subunit IFNAR-1, while maintaining the strong interaction with the interferon receptor subunit IFNAR-2. This is illustrated with several mutants in a newly characterized site (hereafter termed “Site 1B”) on IFN-α2b that is involved in binding to IFNAR-1. It is also illustrated with several IFN mutants that combine alterations in amino acids found in the newly discovered Site 1B with other mutants in the previously described Site 1A. These antagonists are specific examples of the claim that antagonists for human Type I interferons can be created by altering, singly or in various combinations, the amino acid sequence of amino acids contained in the binding sites for IFNAR-1 The main characteristic of these antagonists is that they have a loss of normal biological activity resulting from an altered site of binding to the receptor subunit IFNAR-1, but they retain strong binding to the receptor subunit IFNAR-2. Because of the strong conservation of amino acid sequences among all human Type I interferons, such as IFN-β, IFN-ω, IFN-ε or IFN-κ, analogous antagonists can be derived from other human Type I interferons by creating IFN variants with sufficiently decreased binding to the IFNAR-1 binding, region, while maintaining or enhancing the binding strength to the IFN receptor subunit IFNAR-2. Because of the strong conservation of amino acid sequences among Type I interferons of various animal species, such as human and mouse, it is expected that Type I interferon antagonists appropriate for other organisms can be generated by eliminating biological activity of the IFN by altering the amino acids in the equivalent region of these non-human Type I interferons that are involved in binding to the receptor subunit IFNAR-1, while maintaining or improving the binding affinity for the receptor subunit IFNAR-2. An analogous strategy can also be applied to novel synthetic or chimeric Type I interferons. These Type I IFN antagonists can also provide the basis for the design of peptidic or non-peptidic mimetics that act as Type I interferon antagonists.

A strategy has been adopted for creating inhibitors of native interferons by designing and producing novel interferons that bind with high affinity to IFNAR-2, but have reduced or no measurable affinity for IFNAR-1. Thus, the IFN antagonist(s) (indicated here as IFN*) will bind to IFNAR-2 and interfere with the binding of native or endogenous IFNs, but the bound IFN* will not efficiently bind and recruit IFNAR-1 into the complex, resulting in no signaling and biological effect. The approach requires that the binding site on IFN for IFNAR-1 be identified and modified so that it becomes inactive in binding IFNAR-1. In addition, it is useful to make the binding to IFNAR-2 as strong as possible to interfere with the binding of native IFNs. In principle, any modification or combination of modifications of the IFN structure that sufficiently decreases the interaction with IFNAR-1, without simultaneously decreasing the necessary strong binding to IFNAR-2, can be an antagonist of IFN action. As a second design feature, any modification that increases the strength of binding to IFNAR-2 should increase the effectiveness or potency of the antagonist.

The atomic structures for several Type I interferons have been determined by X-ray crystallography and/or nuclear magnetic resonance. The structure of the prototypic human interferon IFN-α2b has been determined by both techniques and the structural coordinates are available in public databases. Furthermore, work involving site-directed mutagenesis and NMR studies of IFNAR-2 and the complex of IFN-α2b and IFNAR-2 have been used to determine the site on Type I interferons, particularly IFN-αs, for binding to the receptor subunit IFNAR-2, it has also been demonstrated that the affinity for IFNAR-2 varies for different Type I interferons, and the substitution of some amino acids for others can be used to increase or decrease the affinity of IFN and IFNAR-2.

Identification of the site on IFN for binding IFNAR-1 was achieved by making mutants in various amino acid residues of IFN by standard means of molecular biology, and then testing these mutants for three basic properties: (1) biological/biochemical activity: antagonists should have very low or no biological/biochemical activity; (2) binding to the receptor: molecules should have very low affinity for IFNAR-1 but high affinity for IFNAR-2; (3) in biological/biochemical assays of a mixture of an antagonist (IFN*) and an unmodified, active Type I interferon, the antagonist, at some ratio to the unmodified agonist, should be capable of blocking the activity of the unmodified (native) IFN.

Two such antagonist mutants molecules derived from human IFN-α2b are illustrated here; however, other molecules with similar antagonist properties can be created by mutating one or more amino acids in the IFNAR-1 binding region of Type I IFN. Furthermore, this principle for creating Type I IFN antagonists is demonstrated here for human IFN-α2b, but analogous antagonists can be made for other Type I interferons originating from human, mouse or other species.

The two antagonist proteins specifically described here are derived from the protein human IFN-α2b. The so-called “mature” form of this protein has 165 amino acids, numbered by convention from the amino terminus (#1) to the carboxyl terminus (#165) of the protein. Previously published work had identified several amino acid positions in the region of 120-125 where amino acid substitutions either decreased the activity or changed the species specificity of the interferon (e.g., activity of human interferon on mouse cells, etc.). Most of these studies preceded detailed information on the molecular structure of interferon and/or the structure of the receptor, and therefore could not be unambiguously interpreted in terms of receptor interactions: in addition, these studies did not point to the possibility that similar mutants could be used as antagonists.

Various mutations have been made in a group of positively charged amino acid residues that form a cluster on the surface of IFN-α2b, namely: Arg120, Lys121 and Gln124. From these mutations, two interferon IFN-α2 variants have been identified that are antagonists of native IFN-α2 activity: (1) a variant with a substitution of glutamic acid (Glu) for arginine (Arg) at position 120 (Arg120Glu); (2) a variant with 2 substitutions: the substitution of glutamic acid (Glu) for arginine (Arg) at position 120 (Arg120Glu) combined with the substitution of glutamic acid (Glu) for lysine (Lys) at position 121: IFN-α2b-[Arg120Glu/Lys121Glu]. Using the one-letter code for amino acids, these two protein antagonists can be designated: IFN-α2b[R120E] and IFN-α2b[R120E/K120E]. These represent only two possible substitutions in the amino acids that form the binding site for IFNAR-1 that produce an antagonist.

### Type I Interferon and Approach to a Type I IFN Antagonist

Type I interferons were originally discovered, as secreted proteins that have strong and broad-spectrum antiviral activity. It has since been recognized that (1) interferon is actually a family of related proteins; and (2) the biological effects extend far beyond the direct promotion of an “antiviral state” in target cells.

In addition to their direct antiviral and, for some cells, antiproliferative effects, the Type I IFNs have widespread effects on most cells of the immune system, and hence are considered important molecules in linking early or innate immune responses to infection with later immune adaptive responses. Among the known effects of Type I IFNs is the induction of MHC (HLA) class I molecules. Type I IFNs act on a number of cells to modulate the production of other cytokines, chemokines and cellular recognition molecules, which serve to mediate many immune effects; conversely, other cytokines can modulate the production of Type I IFNs. While plasmacytoid dendritic cells are major producers of IFNs, the IFNs also promote the differentiation of monocytes into monocytic or common dendritic cells, the major antigen-presenting cell, with concomitant changes of the cytokine profile for these cells. Type I. IFNs are also major activators of natural killer (NK) cells and CTLs, both of which act on virus-infected cells, or cells with other intracellular pathogens. IFNs can also serve as survival factors for both CD4+ and CD8+ T cells. IFNs have direct effects on B-cell maturation and immunoglobulin class-switching, and act on B cells through the antigen-presenting activity of dendritic cells. Thus, the effects of Type I IFNs on the immune system are widespread and generally reinforcing toward the mobilization of a coordinated defense against viruses and possible other intracellular pathogens, such as Listeria monocytogenes.

Although the major role of Type I IFNs is a potent physiological antiviral proteins, the additional immunomodulatory role of Type I interferons is believed to be the basis for the recently described role for IFN-αs in the pathogenesis of systemic lupus erythematosus, Sjögren's syndrome and possibly other autoimmune diseases syndrome. This has stimulated interest in the development of antagonists or blockers of Type I IFN action. Considering the complex pleiotropic effects of IFN in the immune system, other therapeutic indications for IFN antagonists may be expected. Moreover, in animal models and for in vitro experiments, there is a need for potent IFN antagonists.

### Type I IFNs and their Receptor Interactions

For mammals, the Type I interferons thus far always include the IFN-α and IFN-β subtypes and may include other subtypes, such as: IFN-ω, IFN-κ, IFN-ε, IFN-τ, IFN-ζ/limitin, IFN-δ and the newly discovered IFN-ν. Humans express multiple Type I IFNs (13 IFN-αs and 1 each of IFN-β, IFN-ω, IFN-ε and IFN-κ); for some of the human IFN-αs, there are allelic variants. The human IFN-αs are highly related in amino acid sequence and structure, with 80-98% amino acid identity. In pair-wise comparisons of the amino acid sequences of the human Type I IFNs, they can display as little as about 25% amino acid sequence identity, but many of the non-identical positions of the protein that show amino acid sequence variation have limited variation, often with similar amino acids occupying equivalent positions. The strong relatedness of the Type I IFNs is seen in their three-dimensional structure, which has been determined by X-ray crystallography and/or nuclear magnetic resonance for human IFN-α2b and IFN-α2a, murine and human IFN-β and ovine. IFN-τ. The type I IFNs have highly homologous 3-dimensional structures, based on a bundle of 5 α-helices and connecting loops; these are labeled Helices A-E, with the loops labeled according to the helices they connect (e.g., the “AB loop”, which connects helix A to helix B).

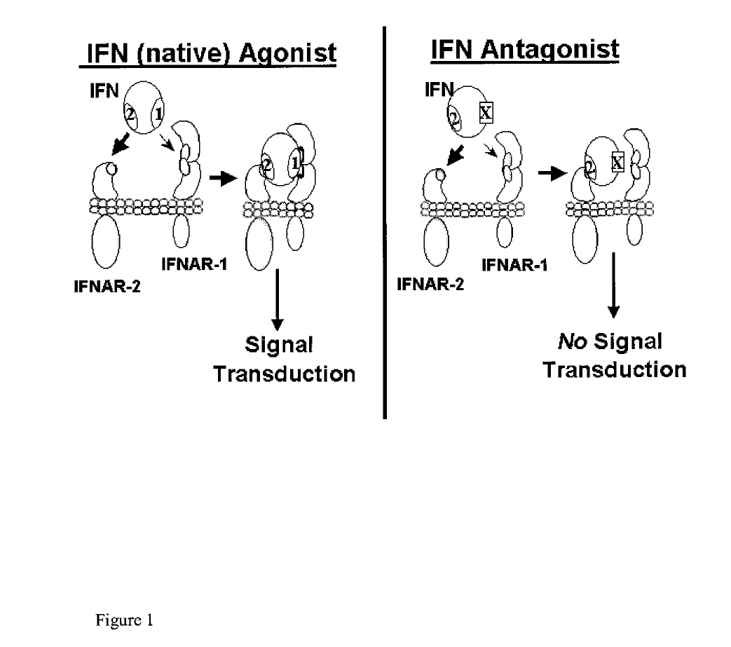

A defining characteristic of the Type I IFN family is that they exert their biological effects through a common high-affinity receptor, IFNAR, composed of 2 transmembrane protein subunits, IFNAR-1 and IFNAR-2 (FIG. 1). The subunits of IFNAR make distinct contributions to ligand binding. Human and mouse IFNAR-1 have low but varied intrinsic affinity for the various IFNs (K_{d}˜0.05-5 μM), whereas IFNAR-2 has moderate to high affinity for IFNs (K_{d} 0.1-100 nM). There is evidence that IFNs bind to the receptor through a sequential binding mechanism, whereby IFN binds first to the high-affinity IFNAR-2 to form a binary complex, which then recruits IFNAR-1 into an active IFNAR-2:IFN:IFNAR-1 complex of stoichiometry 1:1:1. When taken together, as on the cell surface, the receptor complex binds ligand more tightly, increasing its affinity 3-10-fold over IFNAR-2 alone. Assembly of the tertiary complex leads to intracellular signaling and the various biochemical and physiological effects of IFN (FIG. 1).

Prior knowledge of the interactions of IFN with its receptor is extensive but was not sufficient for the design of the IFN antagonists described here. Although many structure/function and mutagenesis studies of IFNs were conducted prior to 1993, interesting amino acid positions on IFN could not at that time be assigned definitive functional roles nor could they be understood in terms of specific interactions with the receptor subunits (IFNAR-1 was discovered in 1990, and IFNAR-2 was identified in 1994).

Human interferon IFN-α2 is probably the most commonly used interferon for experimentation. It has high biological activity, is the basis for many interferon therapeutics, and is the reference molecule used in these experiments. Therefore, the amino acid sequence of the allelic form denoted IFN-α2b is presented here (the allelic form IFN-α2a differs at position 23 by the substitution of Lysine for the Arginine of IFN-α2b). As explained above, most mature human IFN-αs have 166 amino acids, while IFN-α2 has 165, with an apparent deletion of 1 amino acid corresponding to amino acid #44 in the alignment of the family of human IFN-αs.

The amino acid sequence of the mature IFN-α2b protein, lacking the signal peptide required for secretion from mammalian cells, is

| | |
|---|---|
| 1 | CDLPQTHSLG SRRTLMLLAQ MRR1SLFSCL KDRHDFGFPQ EEFGNQFQKA |
| 51 | ETIPVLHEMI QQIFNLFSTK DSSAAWDETL LDKFYTELYQ QLNDLEACVI |
| 101 | QGVGVTETPL MKEDSILAVR KYFQRITLYL KEKKYSPCAW EVVRAEIMRS |
| 151 | FSLSTNLQES LRSKE - 165 |

When the protein is expressed in E. coli, as in the current experiments, the natural N-terminal cysteine (C) may be preceded by a formyl-methionine (fMet) residue. However, in various investigations of the structure and function of interferons, this modification, deriving from its expression in E. coli, seems to have no significant effect on the functional properties of the molecule.

The interaction of IFN-αs with IFNAR-2 is well defined. The affinity varies for the different Type I IFNs, with most affinities in the range of 1-10 nM. The molecular interactions have been defined most specifically by the complementary tools of functional mutagenesis of IFN-α2 and IFNAR-2, and by structural studies by NMR, which produced both the structure of IFNAR-2 and an independent determination of residues at the binding site. On IFN-α2, the key residues for interacting with IFNAR-2 form a contiguous patch, contributed by residues from the A helix, AB loop and F helix. Recent NMR experiments confirmed many of these residues of IFN-α2, but implicated several additional residues in the same surface patch of IFN-α2; thus, the structural studies and mutagenesis studies provide complementary information. The interaction face for IFN-α2 on IFNAR-2 is complementary to the site on IFN-α2. It was also shown that the C-terminal 8 amino acids of IFN, which show considerable variation among Type I IFNs, can modulate the affinity for IFNAR-2 by a factor of 20-fold. These studies provide a fairly complete description of the IFNAR-2/IFN-α2 interaction. The results are also consistent with earlier mutagenesis experiments, including those with IFN-β. The residues of IFN-αs and other Type I interferons that interact with receptor subunit. IFNAR-2 will hereafter be collectively called. “Site 2”.

Knowledge of the interactions between IFNs and IFNAR-1 is less extensive, and the current investigations provide important new information that enables the novel interferon antagonists described here. Our understanding of the interactions between Type I interferons and IFNAR-1 have been limited by: (1) the larger size of IFNAR-1, which makes it a larger project for mutagenesis studies; (2) the lower affinity of the interactions between IFNAR-1 and interferons that are more difficult to measure; and (3) the lack of experimentally determined three-dimensional structures of IFNAR-1 and the IFNAR-1/IFN complex, probably resulting from both the size of IFNAR-1 and the weakness of the binding of IFNs to IFNAR-1.

One site on IFN-α for binding IFNAR-1 was previously identified, and the importance of this interaction for differential Type I IFN biological effects was demonstrated. Residues and regions on the B and C helices that are important for IFN-α2 binding to IFNAR-1 were also identified. Although no residues were found whose substitution by alanine had dramatic (10-fold) effects on receptor binding or activity, a cluster of residues on the surface of IFN-α2 was identified, including F64, N65, T69, Y85, and Y89 that, when mutated individually to alanine, decreased binding to IFNAR-1 by 3-to-5-fold. (A single L80A mutant, located slightly away from this cluster showed similar effects, for reasons that are not understood.) When combined, the L80/Y85/Y89 alanine triple mutant had only 3% potency in an antiviral assay and 0.6% in an antiproliferative assay, while the simultaneous substitution of alanine for the 4 residues of IFN-α2b at N65/L80/Y85/Y89 produced a protein with <1% antiviral activity and <0.1% antiproliferative activity, relative to the native IFN-α2. However, even with these four alanine substitutions in 1 contiguous patch, there was residual, albeit low, biological activity. In contrast to these decreases in activity, alanine substitutions for the neighboring triad of H57, E58 and Q61, increased the affinity for IFNAR-1, with increases in both antiviral and antiproliferative activity. Biophysical measurements demonstrated that alanine substitutions at H57, E58, Q61, F64, H65, L80, Y85, Y89 affected binding to IFNAR-1 but not to IFNAR-2. It was also showed that increasing the affinity for IFNAR-1 by making a triple alanine substitution at H57/E58/Q61 could dramatically increase the affinity for IFNAR-1, the consequent increases in anti proliferative and other activities. Other genetically engineered mutants in the H57/E58/Q61 sequence with higher affinity for IFNAR-1 displayed higher antiproliferative and antitumor activity. Other research has implicated some of these residues in IFN biological activity and/or receptor binding, but without the confirmation that these residues interact specifically with IFNAR-1. Recent research definitively shows that this site, including amino acids at positions 57, 58, 61, 64, 65, 85 and 89, represents an interaction site with IFNAR-1. This site is referred to as “Site 1A”.

The mode of IFN-mediated receptor activation suggests several possible types of IFN antagonists. These include anti-IFN antibodies, anti-receptor antibodies, and recombinant-DNA derived soluble fragments of the IFNAR-2 receptor subunit (“receptor decoy”). However, the strategy documented here is the development of an IFN analogue that blocks the normal biological activity of native Type I interferons. The antagonistic IFN analogue binds strongly to IFNAR-2 (and can therefore block the binding of native IFNs to IFNAR-2), but doesn't bind productively to IFNAR-1 (FIG. 1). Thus, this antagonist will form a “dead-end” IFN/IFNAR-2 binary complex, and will not mediate the formation of a productive IFN/IFNAR-2/IFNAR-1 ternary complex that initiates cellular signaling and biological activity. Such an analogue can be created by modifying the IFNAR-1 binding site on a Type I IFN to eliminate effective binding to IFNAR-1 (FIG. 1).

This design strategy is analogous to the strategy utilized for creating antagonists to several other cytokines, such as granulocyte-macrophage colony-simulating factor (GM-CSF) and human growth hormone in which one of the two receptor sites on the cytokine is disabled. However, this approach has not previously been applied to the development of Type I interferon competitive antagonists, nor were there identified appropriate variants in Type I IFNs with sufficiently low or no detectable biological activity due to sequence variation in the IFNAR-1 binding site. Therefore, the work that is the subject of this application is novel at least by virtue of: (1) identifying and characterizing a new site on Type I IFNs involved in IFNAR-1 interactions; and (2) demonstrating that mutants or variants of the IFNAR-1 site act as IFN antagonists; and (3) the construction of IFN-α2 analogues with in vitro antagonist activity.

### EXAMPLES

### Identification and Characterization of a Second IFNAR-1 Binding Site on Human IFN-0

Although multiple-site mutants in Site 1A of IFN-α2 show strongly decreased biological activity and binding to IFNAR-1, it has been demonstrated that the relative importance of Site I A for receptor binding and biological activity may vary for different IFN-αs. Specifically, mutants in the hybrid human-derived interferon IFN-α2/α1, composed of the first 61 amino acids of human IFN-α2 (amino acids 1-61) and the next 104 amino acids derived from human IFN-α1 (corresponding to residues 63 to 166 of mature IFN-α1) did not show strong decreases in activity when amino acids in Site 1A were mutated to alanine (Table 1). This human hybrid IFN has the unusual property of having high biological activity on cells of human, murine and other mammalian species origins. In our experiments with the hybrid IFN-α2/α1, when alanine was substituted at residues F64, N65, L80, C85 (homologous ot Y85 in IFN-α2), Y89, singly or in groups, there was little effect on biological activity on either human or murine cells, in dramatic contrast to the results from IFN-α2 (Table 1).

TABLE 1
Antiviral Activity of IFN-α Alanine Mutants in Receptor Site 1A

| | Antiviral |
|---|---|
| | Activity (%) |
| IFN-α2/α1 variants | |
| Wild-type | 100 |
| L80/C85/Y89 | 54 |
| N65/L80/C85/Y89 | 63 |
| IFN-α2 variants | |
| Wild-type | 100 |
| L80/Y85/Y89 | 3.5 |
| N65/L80/Y85/Y89 | 0.9 |

Table 1. Antiviral activity of IFN-α2 and IFN-α2/α1. Values for IFN-α2 from Schreiber et al., confirmed by M. Pan and J. A. Langer (unpublished).

For the chimeric IFN-α2/α1, even the 4-site alanine substitutions (N65/L80/C85/Y89) and the 5-site alanine substitution mutant (F64/N65/L80/C85/Y89) showed only 2-to-3-fold decreases in antiviral activity. Also demonstrated was that Site 1A mutants of IFN-α2/α1 do not show strong decreases in binding affinity to IFNAR-1, while the Site 1A mutants of IFN-α2 do. It is therefore concluded that Site 1A is much less important for binding IFNAR-1 in IFN-α2/α1 than in IFN-α2. This suggests that there are other residues or another IFNAR-1 binding site on IFN-α2/α1, and possibly on all Type I IFNs.

A second site on Type I IFNs that contributes strongly to binding to IFNAR-1 has been characterized. This region is denoted as “Site 1B”. Data demonstrates that amino acids within this site are critical for the binding of both the hybrid IFN-α2/α1 and for IFN-α2. As described below, mutations in some amino acids of this site lead to virtually complete loss of antiviral activity. These variants act as novel competitive antagonists of native Type I interferons.

To localize a putative second site on Type I IFNs for binding to IFNAR-1, it is noted that previous but sometimes contradictor); work using various Type I interferons had suggested that mutation of some amino acids on the D-helix can lead to changes in biological activity, presumably resulting from altered binding to the interferon receptor. For instance, a region of IFN-α2 was identified as being involved in the cross-reactivity of HuIFN-αs with murine cells, with strong decreases in antiviral activity noted from charge reversal of Arg120 to Glu and the change of Gln124 to Arg. However, for HuIFN-β, alanine substitutions of K123 and R124 (equivalent to R120 and K121 of IFN-α2) produced only modest effects. In none of these instances was biological activity completely lost. In the IFN structure, these residues constitute a positively-charged patch (in IFN-α2: R120,K121,Q124; and in IFN-α1: K121,K122,R125); this site is relatively far from the identified IFNAR-2 binding site (“Site 2”; above); more recent research suggests the likelihood that these mutations might contribute to binding IFNAR-1.

We have systematically examined the contribution of the D-helix by mutating residues of the D-helix on IFN-α2 and/or on IFN-α2/α1 (Table 2, Table 3).

TABLE 2
Alignment of amino acid sequences of Helix D

| | |
|---|---|
| IFN-α2b (start residue #114) | 114-DSIL₁₁₇AVR₁₂₀KYFQRITLYLKEK |
| IFN-α2/α1 (start residue #114) | 114-DSIL₁₁₇AVK₁₂₀KYFRRITLYLTEK |

TABLE 3
Antiviral activity (relative %) of mutants of IFN-
α2/α1 and IFN-α2 (partial results)

| | |
|---|---|
| IFN-α2/α1 | IFN-α2 |

| | Human | Murine | Human |
|---|---|---|---|
| IFN Mutant | HeLa | L929 | HeLa |
| Wild-type (native) | 100 | 100 | 100 |
| R120A | nd | nd | 4 |
| R120A/K121A | 8.3 | 33 | 2 |
| R120E | <0.04 | 0.04 | <0.028 |
| R121E | 8 | 0.5 | nd |
| Q124E | 20 | 100 | nd |
| R120E/K121E | <0.08 | ≦0.04 | <1.3 |
| R120E/K121E/Q124E | <0.04 | ≦0.01 | nd |
| L117A | nd | nd | 27 |
| L117A/120A | nd | nd | <0.17 |
| L117A/R120A/K121A | nd | nd | <0.04 |
| A4 (N65/L80/Y85/Y89)A | 63 | nd | 3.3 |

Specification of “<” indicates that no activity was tested at the current upper limits of the protein concentrations tested. “nd” = “not done”

The most important results are summarized in Table 3. In a duster of strongly conserved basic amino acids (positions 120, 121, 124 and 125), mutagenesis involved charge-reversal (Arg or Lys to Glu) or less dramatic substitution by alanine (Table 3). The magnitude of effects for charge-reversal mutants in IFN-α2/α1 was: R120>K121>Q124 (mutations in R125 showed minimal effects; results not shown) Importantly, the single-site charge-reversal mutation Arg120Glu (R120E) causes total loss of activity for both IFN-α2/α1 and IFN-α2 Moreover, the IFN-α2/α1[R120E] mutant had not detectable activity on either human or marine cells (Table 3), demonstrating the conservation of sequence and function between the human and murine models. Mutation to alanine of the conserved Leucine 117, adjacent to R120, which has not previously been investigated, also showed a modest decrease in activity. However, when L117A is combined as a double mutant with R120A (L117A/R120A) or as a triple mutant with R120A/K121A (L117A/R120A/K121A), the antiviral activity of the double and triple mutants decreased below the detectable limits (Table 3). For all Helix D mutants tested to date, where antiviral, activity was low or undetectable, the binding affinities for IFNAR-1, as measured by surface plasmon resonance, is below the detectable limits of the technique (data not shown); however, as predicted, the binding affinities for IFNAR-2 are normal (K_{d} 1-3 nM; data not shown), demonstrating that the change in activity is, in fact, due to changes in the interaction with IFNAR-1 and that the proteins do not suffer from global folding defects.

Considering the physical separation of these residues from receptor binding Site 1A, it is reasonable to conclude that amino acids Leu117, Arg120, and Arg/Lys121 are part of a critical IFNAR-1 binding site on Type I IFNs (“Site 1B”), distinct from the previously identified Site 1A, Appropriate mutations in the amino acids of this site can produce interferon variants with reduced or no biological activity, and with reduced or no detectable binding to IFNAR-1, but with binding to IFNAR-2 comparable to that of native Type I IFNs. However, since cytokine/receptor interactions often involve an extensive region of contact between the proteins, it is likely that other neighboring amino acids may also be part of Site 1B. Thus, the full characterization of Site 1B awaits further studies by mutagenesis and by physical structural studies by X-ray crystallography or NMR. Nevertheless, the finding that mutations in this region

can decrease IFN activity of IFN-α2 and/or IFN-α2/α1 to undetectable levels provides a basis for designing interferon antagonists.

### Description of Type I IFN Antagonists

Amino acid residues of IFN-α2 that are part of the binding site for IFNAR-1 have been identified and IFN-α2 variants in the IFNAR-1 site have been tested as prototype Type I IFN antagonists. The IFN-α2 analogues have the following properties: (1) they are deficient in normal activities associated with Type I interferons (2) they are deficient in productive interactions with IFNAR-1; (3) they retain the ability to bind to IFNAR-2; and (4) they block the biological activity of normal Type I interferons in one or more assays.

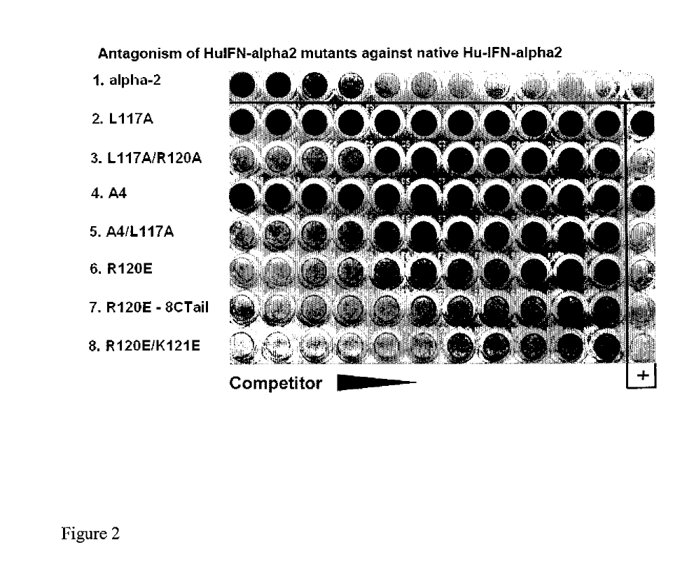

IFN-α2 mutants with no detectable antiviral activity were examined for their ability to antagonize the antiviral activity of native IFN-α2 in a viral inhibition assay. The viral inhibition assay (“antiviral assay”; “cytopathic effect assay”) is the standard assay for determining the potency of interferons; blocking IFN activity in this assay is a standard test of potentially antagonistic materials (e.g., antibodies). The assay is extremely sensitive to interferon activity. In the current version, test cells are incubated with IFN in a 96-well plate format overnight at 37° C. in order to develop protection against virus infection. Then a cytopathic virus is added. After a suitable period to permit the virus to kill cells (in this case 24-30 hours), plates are stained with a dye that reveals the presence of live (i.e. IFN-protected) cells in the variation of the assay employed here, serial dilutions of putative antagonists are incubated with the active IFN-α2 and cells to determine whether the mutant IFNs will block the protective effect of the active IFN-α2. Antagonism of the protective IFN effect is manifested in cell killing, and a lack of staining in the wells of the plate (FIG. 2).

Sample data for some mutants is shown in FIG. 2. The top row (“alpha-2”) shows 2-fold serial dilutions of the active IFN-α2 and demonstrates the ability of the test amount of active IFN-α2 to protect human HeLa cells after challenge with the vesicular stomatitis cytopathic virus (VSV). The remaining 7 rows show the effect of different mutants on the ability of constant amounts of native IFN-α2 to protect. HeLa cells from VSV, where the mutants are added at a high concentration in Well 1 (left side) of each row, and then at decreasing concentrations across the row. The final column of each row has no native IFN-α2, but only a high concentration of the mutant to determine whether the mutant itself retains any antiviral activity. Row 2 demonstrates that the IFN-α2[L117A] mutant does not block the action of IFN-α2; on the contrary, as shown in the last column of Row 2, the IFN-α2 [L117A] mutant, in the absence of native IFN-α2, can protect HeLa cells from VSV. However, when the double mutant L117A/R120A is tested (row 3), the mutant at high concentrations antagonizes the ability of IFN-α2 to protect the HeLa cells, i.e., the cells are not protected by native IFN from the VSV challenge virus, and are killed. As the mutant IFN is diluted, moving across the Row 3 toward the right, the blocking effect of the mutant is lost, and the protective effect of the native IFN on the cells is seen by the staining of the cells. Similarly, in Rows 6 and 8, the single-site mutant R120E and the double-site mutant R120E/K121E display antagonist activity.

As mentioned above, the 4-site alanine substitution mutant in Site 1A (“A4”=N65A/L80A/Y85A/Y89A) has low but measurable biological activity; this is shown in Row 4 (“A4”), in the right-most well, and this mutant is unable to antagonize active IFN-α2. However, as seen in Row 5, when the mildly active Site 1B L117A mutation is introduced into the A4 molecule, the resulting 5-site mutant (“A4-L117A”; Row 5) has no detectable biological activity (see column 12); and can block the activity of native IFN-β2. Thus, mutants in Site 1A with reduced biological activity can interact with mutations in Site 1B to form effective antagonists. The antiviral assay is a stringent test of antagonism because any active IFN that interacts productively with the receptor during the initial overnight incubation will lead to cellular protection against subsequent virus infection A list of some mutants that act as antagonists in the antiviral assay is found in Table 4.

TABLE 4
Antagonism by Mutants of IFN-α2

| Site 1A | Site 1B | | Antiviral |
|---|---|---|---|
| Substitutions¹ | Substitutions | Antagonist | activity (%) |
| — | R120A | No | 4 |
| — | R120E | Yes | no |
| — | R120E-8CT² | Yes | no |
| — | R120A/K121A | No | 2 |
| — | R120E/K121E | Yes | no |
| — | L117A | No | 27 |
| — | L117A/R120A | Yes | no |
| — | L117A/R120A/K121A | Yes | no |
| A4¹ | — | No | 3 |
| A4¹ | L117A | Yes | no |
| A4¹ | R120A | Yes | no |

A partial list of mutants, with their antiviral activity and their ability to antagonize the activity of IFN-α2 in the antiviral assay. Antiviral activity is taken as a percentage of that of native IFN-α2 measured on human HeLa cells, with a VSV challenge virus (data from Table 3).
¹The designation of “—” denotes that there are no mutations in the amino acid residues implicated in the IFN site corresponding to Site 1A. “A4” denotes the 4-site alanine substitution mutant (also denoted “NLYY”) N65A/L80A/Y85A/Y89A.
²R120E-8CT is an IFN-α2[R120E] mutant which also has a modification at the C-terminus, where amino acids at the C-terminus of IFN-α8 have been substituted for the equivalent amino acids in IFN-α2, which has been reported to increase the affinity for the IFNAR-2 receptor subunit.

Some mutants were also tested in an assay for the ability of IFN to activate the Stat1 latent transcription factor; these results were generally consistent with the antiviral assays (data not shown). Cells are incubated with native IFN-α2 alone, or with IFN-α2 in the presence of the IFN-α2 mutants, to test whether the latent transcription factor, Stat1, is activated. This is tested by the ability of the activated Stat1 to interact with a radioactively labeled oligodeoxynucleotide (the “probe”) and to shift the migration of the probe in an electrophoretic gel (“electrophoretic mobility gel-shift” assay; EMSA). Consistent with the antiviral activity results, the R120A/K121A mutant retains some activity to activate Stat1, while the charge-reversal mutants R120E and R120E/K121E do not activate Stat1 Moreover, the presence of the mutants R120E and R120E/K121E blocks the ability of native IFN-α2 to activate Stat1. That is, in the Stat1 activation assay, the R120E and R120E/K121E mutants are IFN antagonists. The Stat1 activation assay also showed antagonist activity for the mutants L117A/R120A, A4-1117A, with partial antagonism activity for the A4 mutant (data not shown).

### Further Development and Extensions of the Type I IFN Antagonists

Further development of the novel Type I IFN antagonists includes improvements such as increasing the affinity for the IFNAR-2 receptor subunit. This enhancement will increase the biological potency of these molecules and permit antagonism at lower concentrations of antagonist, and therefore will permit in vivo and therapeutic testing and use of these or similar antagonists. This increased affinity can be achieved by any of several standard approaches, including site-specific mutagenesis of amino acids known or suspected to be involved in binding IFNAR-2. Alternatively various site-directed random-substitution techniques or individual amino acids or groups of amino acids in the IFNAR-2 binding site can be used. Examples of such techniques include phage display and ribosome display.

The novel antagonists demonstrated here are based on the amino acid sequence of IFN-α2b, and antagonism against native human IFN-α2 was demonstrated. However, the strong evolutionary conservation of Type I interferons observed in the homologous three-dimensional structures and in the amino acid sequence relationships, together with the fact the Type I IFNs, by definition, act through the common IFNAR receptor; justifies the logical extension of this work, including: (1) Human antagonists derived from IFN-α2b should serve as antagonists for all other human Type I IFNs, (2) Other human Type I IFNs, including the other IFN-α's, IFN-β, IFN-ε, IFN-κ and IFN-ω, as well as synthetic and chimeric human IFNs, can be used as the basis for human Type I IFN antagonists by changing appropriate amino acids in the homologous sites for binding IFNAR-1. Many of these amino acids are identical or similar to those identified for the IFNAR-1 binding site of IFN-α2; (3) Because of the strong amino acid sequence similarity between the human Type I IFNs and those of other mammalian species, analogous antagonists can be readily derived from non-human Type I IFNs for use on cells or in animals of other species, such as mice, rats, cows, and monkeys. This is supported by the observation that the IFN-α2/α1[R120E] mutant has no detectable antiviral activity on either human or murine cells (Table 3). (4) Knowledge of the IFNAR-1 binding site of human Type I IFNs can form the basis for the design and/or selection of peptide or non-peptidic mimetic antagonists.

These human-derived Type I IFN antagonists can be effective therapeutic agents in human conditions such as systemic lupus erythematosus and Sjögren's syndrome that involve the dysregulation of Type I interferons. These molecules also have application in in vitro experiments Analogous non-human Type I IFN interferons based on this strategy will similarly be useful to block interferon effects in tissue culture or in vivo situations of the appropriate animal species.

### Comparison with Other Types of Type I IFN Antagonists

In principle, Type I IFN action in vivo can be inhibited at any step of the “interferon cycle”, from the production of native Type I IFN from appropriately stimulated cells to the intracellular signaling pathways initiated by Type I IFNs. The use of IFN analogues as antagonists proposed here differs in essential ways from other classes of antagonists of interferon action including: specific oligodeoxynucleotides (ODNs) that inhibit the production of IFN by IP N-producing cells; neutralizing antibodies to IFN-α antibodies to the IFN receptor that inhibit the binding of IFN to its receptor; soluble receptors based on IFNAR or other IFN-binding molecules circumstances; inhibitors of intracellular signaling by IFN. All methods of systemic IFN blockade are likely to cause increased viral susceptibility, at least temporarily. However, close monitoring and classic antiviral therapeutics may permit management of this susceptibility for short-term to moderate-term application.

A full comparison of potential advantages/disadvantages of the IFN-based inhibitors to each potential alternative strategy is beyond the scope of this invention, and is speculative. However, some of the parameters for consideration are: (1) pharmacokinetic/pharmacodynamic properties; (2) breadth of action against the spectrum of Type I IFNs; (3) mode of delivery; (4) duration of effect; (5) costs; (6) side-effects.

The IFN-based antagonists of this invention are a robust technology for in vivo IFN blockades and for in vitro reagents. Examples of some considerations include the following (I) Based on size considerations and known pharmacokinetics of other active IFNs, the currently described class of antagonists is likely to have relatively short half-lives in vivo (<1 day) compared to the half-lives of the larger monoclonal antibodies and receptor decoys. The immediate consequences may be shorter effective lifetime of effect and of side-effects (e.g., potential susceptibility to viruses during treatment). (2) However, because of the well-developed technologies related to IFNs and cytokines, the half-life of the IFN analogue antagonists can, if desired, be increased by such known technologies as site-specific or non-specific attachment of polyethyleneglycol (“pegylation”), or construction of a fusion protein with human serum albumin, or with the heavy chain of a human immunoglobulin. (3) The IFN-based antagonists, by binding to IFNAR-2, should establish a blockade of all Type I IFNs, including, for humans, other IFN-α's, IFN-β, IFN-ε, IFN-κ and IFN-ω. This is in contrast to anti-IFN-α antibodies that only block Type I IFN action induced by IFN-α's, but which leave uninhibited the potential action of other Type I IFNs. It is unknown which is more advantageous. (4) As an E. coli-derived material, the IFN-based antagonists should be quite competitive on price with other classes of IFN blockades; both as research reagents and as potential therapeutics. If advantageous, however, these antagonists could also be produced in other expression systems, such as yeast, insect cells, mammalian cells, whole plants or whole animals. (5) The IFN-based antagonists can be the basis for thither development of small-molecule mimetic antagonists.

### Construction of IFN-α2b Variants

Variants were produced by standard methods of molecular biology. In the current examples, the DNA sequence was confirmed by DNA sequencing of both DNA strands. The complementary DNA (cDNA) representing the 165-amino acid coding region of IFN-α2b, followed by a codon representing a translational “stop” signal (“TGA”) was supplied by Dr. Sergei Kotenko (UMDNJ-New Jersey Medical School). Human-derived chimeric IFN-α2/α1 (“IFN-αA/D”) cDNA was kindly provided by Dr. Sidney Pestka (UMDNJ-Robert Wood Johnson Medical School) and the cDNA fragment was restricted and cloned into BamHI/NdeI-digested pET-11a vector (Novagen). The DNA sequence was confirmed by DNA sequencing of both DNA strands. Site-directed mutagenesis (Quickchange kit; Stratagene, USA) was used to create a series of human IFN-α2 and IFN-α2/α1 variants.

The expression of heterologous proteins interferons in Escherichia coli is often hampered by the presence of arginine low-usage codons, AGO and AGA. Site-directed mutagenesis was used to construct a series of pET-11a-Hu-IFN-α2b gene variants (Hu-IFN-2b-R331213), which were changed in the DNA nucleotides corresponding to arginine at positions 12, 13 and 33 from codons rarely used in E. coli (AGA/AGG) to codons that occur frequently in E. coll. These comprised the replacement of arginine clusters (Arg¹²Arg¹³ and Arg³³) mutant (pET-11a-Hu-IFN-α-2b-R331213, pET-11a-HuIFN-α2b-R1213) enhanced their expression. It should be emphasized that these changes are not required for this invention: the changes do not modify the properties of the final protein, but only increase the efficiency of producing it for initial study. The plasmid pET-11a-HuIFN-α2b-R331213 is the starting point for making the IFN-α2b variants in amino acid sequence.

Changes in the cDNA and ultimately in the amino acid sequence of IFN-α2 were introduced by the polymerase chain reaction (PCR) with oligodeoxynucleotide primers that correspond to the desired changes in protein sequence. This was accomplished with standard techniques of molecular biology, in this case using a commercial site-directed mutagenesis kit (QuickChange kit; Stratagene, USA). Examples of oligodeoxynucleotide primers for mutagenesis include: (1) For the human IFN-α2b[R120A] mutant: 5′-3′ C TCC ATT CTG GCT GTG GCG AAA TAC TTC CAA AGA ATC and 5′-3′ GAT TCT TTG GAA GTA TTT CGC CAC AGC CAG AAT GGA G; For the human IFN-α2b[R120E] mutant: 5′-3′ C TCC ATT CTG GCT GTG GAG AAA TAC TTC CAA AGA ATC and 5′-3′ GAT TCT TTG GAA GTA TTT CTC CAC AGC CAG AAT GGA G. For the IFN-α2b[R120E/R121E] double mutant, the template was the DNA plasmid for the IFN-α2b[R120E] mutant, using primers: 5′-3′ TCC ATT CTG GCT GTG GAG GAA TAC TTC CAA AGA ATC and 5′-3′ GAT TCT TTG GAA GTA TTC CTC CAC AGC CAG AAT GGA. Other mutagenic primers were similarly designed. All constructs were confirmed by DNA sequencing with an automated DNA system.

After the mutagenic PCR reaction, the plasmids and transformed into E. coli DH5α cells. Cells were grown, plasmids were extracted by the QIAprep Spin Miniprep kit (Qiagen, USA). The presence of the desired mutations in the cDNA was verified by DNA sequencing with an automated DNA system.

### Protein Expression

There are many methods for the expression and purification of recombinant proteins, including recombinant Type I interferons, from E. coli, and the following procedure is one of many possibilities, all of which may provide recombinant proteins with equivalent functional properties. In addition, it is possible to produce the recombinant proteins in various eukaryotic expression systems, including those using yeast cells, insect cells, mammalian cells, whole plants or whole animals.

Plasmid DNAs of IFN-αmutants were individually transformed into E. coli strain BL21 (DE3) Rosetta 2. Bacteria were grown in LB broth containing 100 μg/ml ampicillin at 37° C. overnight. The cultures were diluted 50-fold and incubated at 37° C. with shaking. Protein expression was induced by 0.8 mM isopropyl-β-D-thiogalactopyranoside (IPTG). The bacteria were then grown at the same temperature for 4-6 hr.

The cells were harvested by centrifugation and resuspended in the Buffer A (50 mM TrisHCl pH 8.0, 40 mM NaCl, 5 mM EDTA), Lysozyme (0.2 mg/ml) and 0.2 mM PMSF (Phenylmethylsulfonyl fluoride; Sigma Chemical Co.). The cells were sonicated on ice (3×15 s pulses at 50 W), 1% Triton-X-100 (Bio-Rad) was added to the homogeneous suspension and centrifuged for 20 min at 30,000×g. The pellets were suspended in Buffer A with 0.2 mM PMSF, following sonication and centrifugation as previously described. The inclusion body (IB) pellets were finally resuspended in Buffer A to remove remaining Triton-X-100 and centrifuged at 30,000×g for 20 min. The IB pellet was either solubilized immediately or stored frozen at −80° C. until further use.

The IB pellet was solubilized in 7M GuHCl (guanidinium hydrochloride; Invitrogen) in buffer A overnight at 4° C. with gently shaking. The IB solution was then centrifuged at 30,000×g for 30 min at 4° C. The supernatant was refolded by dropwise dilution into 15 volumes of 0.5.M L-arginine (Sigma), 100 mM Tris-HCl (pH 8.0), 0.2 mM EDTA (pH 8.0) for 24-48 hr at 4° C. The refolded solution was adjusted to 1.6 M (NH₄)₂SO₄ (Sigma, Ultrapure) and centrifuged at 30,000×g for 30 min at 4° C.

The supernatant with refolded protein was loaded on a hydrophobic column (Toyopearl Phenyl-650M; Tosoh Bioscience) which was equilibrated with 0.5 M GuHCl, 50 mM Tris-HCl pH 8.0, 1.6 M (NH₄)₂SO₄. The column was washed with 0.5 M GuHCl, 50 mM Tris-HCl pH 8.0, 1.6M (NH₄)₂SO₄ and 1M urea (Sigma). The proteins were eluted with 0.5 M GuHCl, 50 mM. Tris-HCl pH 8.0, and 1 M urea (Sigma).

The fractions with the IFN were dialyzed against 20 volumes of 20 mM Tris-HCl (pH 8.0), 50 mM NaCl overnight at 4° C. The dialyzed supernatant was applied to a HiTrap Fast-Flow Q-Sepharose ion-exchange column (Amersham Bioscience) for purification, eluting with a linear gradient of 50-500 mM NaCl. Samples were further concentrated and buffer was exchanged to the storage buffer of 20 mM Tris-HCl (pH 8.0), 50 mM NaCl, 0.4 M L-arginine. Purity of proteins was determined by SDS-polyacrylamide gel electophoresis, and the protein concentration was determined by absorbance at 280 nm.

### Cell Culture

Human HeLa and WISH cells were grown in Dulbecco's modified Eagle's medium (DMEM) supplemented with 10% cosmic calf serum (Hyclone) with Glutamax (Sigma). Murine L-929 cells were cultured in Minimum Essential Eagle's Medium with Glutamax at 37° C. and 5% CO₂. NFS-1.0 cells (ATCC #CRL-1705), a murine line that is highly sensitive to the antiproliferative effects of IFN, were cultured in RPMI 1640 supplemented with 15% cosmic calf serum, 5 mM L-glutamine, 1% penicillin-streptomycin (Mediatech, Inc), 1× HEPES buffer (Mediatech), 0.1% 2-mercaptoethanol (Gibco), 2.5 g/L D-glucose (Gibco) and 1 mM sodium pyruvate (Gibco).

### Antiviral and Antiproliferative Assay

Antiviral activity of wild-type and mutant IFNs was assayed as the inhibition of the cytopathic effect of vesicular stomatitis virus (VSV) on human HeLa. WISH or A-549 cells, and with encephalomyocarditis virus (EMCV) on murine L-929 cells. For antiproliferative assays with WISH and L-929 cells, cells (usually ˜1×10⁴) in 50 μL growth medium were added to serial dilutions of IFNs in a 96-well culture dish. Cells were grown for 3-4 days. Medium was removed and cells were stained with crystal violet. Plates were read by eye to find the dilution corresponding to 50% of maximum growth. Alternatively, the crystal violet in the stained cells was solubilized by addition of 100 μL of 50% ethanol/50% Tris-HCl (pH 8.0) (vol/vol). Optical density was read at 586 nm. For WISH cells, the cells released from the plates, and may have undergone cell death during the assay. Data were analyzed by a non-linear fit to a sigmoidal curve and the EC₅₀ and statistical parameters were calculated from the curve using the program “Prism v. 3” (GraphPad, Inc., San Diego).

### Antagonism Assay

Antagonism assays were variations of the antiviral cytopathic effect assay and antiproliferative assays. For the antiviral assay, a constant amount of IFN-α2 (usually 1-2×10⁻¹⁰ M, final concentration; depending on the cell type) was combined with serial dilutions of each IFN variant (the highest concentration of each mutant was in the range of 1-5×10⁻⁶ M, depending on its ability to inhibit IFN activity), Cells were added and incubated overnight at 37° C. and 5% CO₂. VSV was added and the plates were incubated for 24-30 hr. When the cytopathic effect in control wells reached 100%, the plates were stained by crystal violet. In these assays, the active and inactive IFNs could be pre-mixed at room temperature, followed by addition of cells and incubation at 37° C.; i.e., antagonism did not require pre-incubation of cells with the mutant IFN prior to addition of active IFN-α2. Antiproliferative assays were similarly designed, where a constant amount of IFN-α2, sufficient for growth inhibition of each cell line, was added to serially-diluted (usually 2-fold) concentrations of each IFN variant. Cells were added and grown for 3 days. Cells were stained with crystal violet.

### STAT Activation Assay

Another assay of IFN activity and antagonism is the ability of IFNs to activate the intracellular transcription factor STAT-1 in an electrophoretic mobility shift assay (EMSA), or the ability of the variants to interfere with the activation by native IFN-α2. For activation, interferons were added to cells at 0.1 to 10 ng IFN. For antagonism, putative antagonists (1-100 ng) were added to cells in the presence of 1 ng IFN. Cells were incubated for 15 min. at 37° C., extracts were made, and the extracts were incubated with ³²P-labeled oligodeoxynucleotides corresponding to the GAS STAT-1 DNA binding element. Complexes were resolved by electrophoresis on standard polyacrylamine gels, and the gel was autoradiographed.

### Measurement of Binding Affinity

Binding to purified human IFNAR-1 and IFNAR-2 is measured with the Protein Interaction Array system (Bio-Rad) according to published methods. A solution of 0.005% Tween20 in PBS pH 7.4 was used as running buffer at a flow rate of 30 μl/min. For immobilization, an activated EDC/NHS surface was covered with the non-neutralizing antibodies DB2 and 46.10 against IFNAR1-ECD and IFNAR2-ECD, respectively, and blocked with ethanolamine. Thereafter, five of the six channels were reacted with IFNAR1-ECD or IFNAR2-ECD (180 μl at a concentration of 0.5 μM), leaving one channel free as reference. This was followed by cross-linking a second antibody. AA3 for IFNAR1-ECD and 117.7 for IFNAR2-ECD to improve the stability of coupling and reduce leakage of IFNAR2-ECD, without affecting ligand binding. Interferons were then injected perpendicular to ligands, at six different concentrations within a range of 37 to 8,000 nM for IFNAR1 binding and 3.12 to 100 nM for IFNAR2 binding. Data were analyzed with the BIAeval 4.1 software, using, the standard Langmuir models for fitting kinetic data. Dissociation constants K_{D} were determined from the rate constants according to:

$${K}_{D} = \frac{{k}_{off}}{{k}_{o n}}$$

or from the equilibrium response at six different analyte concentration, fitted to the mass-action equation.

### Detailed Results

### Helix D Residues are Important for IFN-α2 Activity and Binding to IFNAR-1.

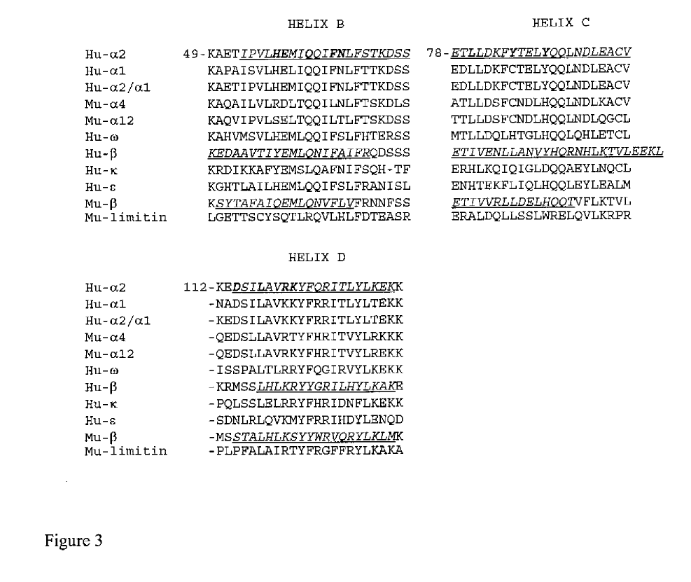

Previous examination of the 13 and C helices of IFN-α2 identified a number of residues that contribute to IFNAR-1 binding. Single-site alanine substitution mutations, however, did not have dramatic effects, and even the 4-site. NLYY mutant (NLYY=N65A/L80A/Y85A/Y89A) of the IFNAR-1 binding region retained about 1% antiviral activity and 0.1% antiproliferative activity. Since other studies had implicated pans of Helix D in IFN activity and possibly in receptor binding, the D-helix of IFN-α2 was examined for residues that might also contribute to IFNAR-1 binding. The D-helix contains several strongly conserved features including a positively-charged patch (in IFN-α2: R120, K121, Q124, R125; although Q124 in IFN-α2 was not examined in this study, the equivalent R124 of IFN-α2/α1 was examined; see below), the conserved Leu 117 and Asp 114 (FIG. 3). This helix is relatively far from the identified IFNAR-2 binding site.

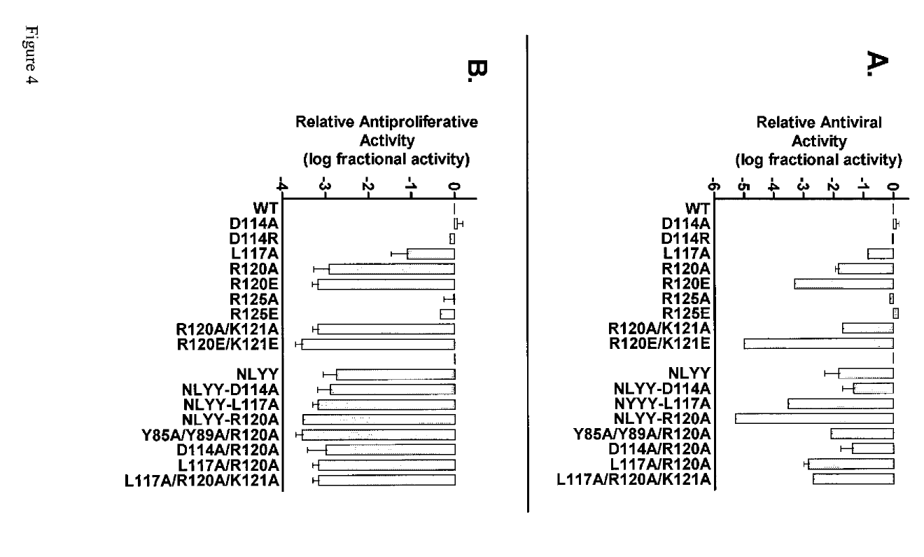

Within the conserved positively charged residues of helix D, substitution of R120 with alanine decreased antiviral activity to 1-3% and reduced antiproliferative activity to about 0.05% of native IFN-α2 (FIG. 4; Table 1A). More dramatic loss of activity occurred with the charge-reversal R120E mutation, where activity was below the threshold of the measurements. Furthermore, when the IFN-α2[R120E] mutation is combined with the carboxyl-terminal 8 amino acids found in IFN-α8 (“120E-8Ctail”), which is reported to increase the affinity for IFNAR-2, the antiviral activity on HeLa cells was still not detected. The two-site it R120A/K121A mutant had similar activity to R120A, suggesting a less important role for K121A, which was not evaluated as a single-site mutant. The R120E/K121E mutant, similar to the single R120E mutant, had no demonstrable antiviral or antiproliferative activity on human cells, and showed a modest (10-fold) decrease of antiviral activity on bovine cells. Moving further along the helix, the charge-reversal R125E mutant had little or no effect on biological activity. Thus, for IFN-α2, substitutions in the positively charged cluster show their relative important in the order, R120>>K121>R125. Leucine 117 is completely conserved in human and murine Type I IFNs, is surface-exposed and is adjacent to R120. Its substitution by alanine decreased antiviral and antiproliferative activity about 5-fold. In combination with R120A, L117A further decreases the antiviral and antiproliferative activity. At the N-terminus of Helix D is Asp 114, conserved in human and murine IFN-αs, but variable in the other Type II IFNs. Its substitution by alanine has no effect on antiviral and antiproliferative activity, measured on WISH cells, and a small effect on antiviral activity measured on HeLa cells. Most single-site mutants retained activity on bovine MDBK cells that are generally highly sensitive to human IFN-αs, and are often less sensitive to modification of HuIFN-α.

As expected, mutations in the D-helix, including those such as R120E/K121E which decreased activity by more than 4 orders of magnitude, did not significantly change the binding affinity for human IFNAR-2 from that measured for native IFN-α2 (K_{D}≈2.5+/−0.5 nM), with almost all mutants being within two-fold of this value (Table 1A). Affinity of R120A for IFNAR-1 and of the 120E-8CTail mutant was decreased by at least 10-fold (“ND”—“not detected”), to the limits of detection of the experimental set-up (K_{D}≧10 μM). Because of technical difficulties, reliable measurements of IFNAR-1 were not obtained for some of the other samples. Nevertheless, the retention of high affinity for if IFNAR-2 excludes direct interactions of helix D residues with human IFNAR-2.

### The Interaction of Site 1A and Helix D for IFN-α2 Activity and IFNAR-1 Binding.

For the IFN-α2 variants, investigations were directed to whether there is a functional relationship between previously identified residues (“Site 1A”) and the functionally important residues on Helix D. Therefore alanine substitutions at each of the residues Asp114, Leu117 and Arg120 were combined with the 4-site alanine mutant in Site 1A residues N65, L80, Y85, Y89 (“NLYY”) (FIG. 4; Table 1A). Although the NLYY mutant has about 1% activity on human HeLa cells, the R120A mutant combined with the NLYY mutants lacks measurable antiviral and antiproliferative activity. Also, the combination of L117A with NLYY significantly reduced activity on human cells from that of NLYY, although the L117A mutant itself only had small effects on biological activity. In contrast, the addition of the D114A mutation to NLYY seems to have little additional effect on the antiviral activity of NLYY on human cells. As with other Site 1A and Helix D mutants, these combined mutants retained affinity for IFNAR-2.

### Helix D Residues are Also Important for IFN-α1/α1 Activity and Binding to IFNAR-1.

To examine residues involved in binding to IFNAR-1 in a different sequence context, homologous mutations were made in Site A and in the D helix in the chimeric interferon IFN-α2/α1. This is a hybrid of human IFN-α2 (amino acids 1-61) and human IFN-α1 (residues 63 to 166). Most of the functionally important residues in the IFNAR-1 site are from the IFN-α1-derived segment of the chimera, rather than from the IFN-α2 N-terminal segment, although many residues are conserved between the two IFNs (FIG. 3). This chimera is particularly interesting in that it has high biological activity on both human and murine cells, as well as those of other species.

In IFN-α2/α1, alanine substitutions in Site 1A residues (N65, L80, C85, Y89), including a 3-site alanine substitution mutant. L80/C85/Y89, and a 4-site alanine substitution mutant (N65/L80/C85/Y89), produced no more than a 2-to-3-fold decrease in antiviral activity on human and murine cells (Table 2A). A 5-site alanine substitution mutant that added Phe64Ala, located in the cluster with N64, C85 and Y89, produced a 10-to-20-fold decrease on the antiviral activity on human cells. A similar trend, but of larger magnitude, was obtained for the antiproliferative effects of IFN-α2/α1 mutants, measured on human WISH cells and murine NFS-01 cells. (The growth of HeLa cells and L-929 cells, used for the antiviral assays, was only weakly inhibited by native IFNs, so WISH cells and NFS-01 cells that responded more robustly to the antiproliferative effects of IFNs were used). Thus, the Site 1A cluster of residues of IFN-α2/α1 has less relative importance for binding to IFNAR-1 than this cluster has for IFN-α2.

Within this region, previous studies of a chimeric IFN-α21/α2c construct suggested that aspartic acid substitution of Cys86 (equivalent to Cys85 in IFN-α2/α1) strongly affected biological activity. However, substitution of Asp for Cys 85 in IFN-α2/α1, either as a single-site mutation or within the context of multi-site mutants, did not markedly decrease antiviral activity on human or marine cells (data not shown). Thus, the effect of substitutions in Site 1A of IFN-αs seems to depend strongly on the IFN-α subtype, i.e., the sequence context.

Contributions of positively-charged D-helix residues of IFN-α2/α1 were more dramatic (fable 3A). In particular, the K120E mutation caused a loss of antiviral activity of >2500-fold on both human and murine cells. Relative antiproliferative activity was at least 10-fold lower than antiviral activity (Table 3A). Thus, this residue is important for both IFN-α2 and IFN-α2/α1, and for the interaction with both human and marine IFNAR-1. However, K120 mutants retained high antiviral activity on bovine MDBK cells, and high affinity for human IFNAR-2, demonstrating that the mutations do not affect global folding of the mutants. For interactions with the human and murine receptor, the positively charged residues have the relative importance: K 120>>K121>R124. Binding to human IFNAR-2, measured by SPR, was similar to the binding of native IFN-α2 and was not affected by these functionally significant mutations (Table 3A). However, binding to human IFNAR-1-ECD was significantly weakened for mutants with lowered biological activity, and was outside the measurement limit (K_{D}>10 μM) for mutants with little or no detectable activity on human cells. (It is also noteworthy that the affinity of IFN-α2/α1 for IFNAR-1 is about 5 times stronger than that of IFN-α2; i.e., the ratio of the K_{D}'s of IFN-α2/α1 to that of IFN-α2 is about 0.2. This stronger binding of the native IFN-α2/α1 to IFNAR-1 makes changes in binding easier to measure, since the affinity is further from the upper limit of affinity of the experimental protocol.)

Mutants in Helix D and Site 1A that Lack Biological Activity are Antagonists of in Vitro Biological activity of IFN-α2.

It is predicted that IFN variants with strong binding to IFNAR-2 and no significant binding to IFNAR-1 should act as competitive antagonists, as has been demonstrated for other cytokines that ligate two receptor subunits to initiate action. As predicted, mutants with no detectable antiviral and antiproliferative activity blocked a protective concentration of human IFN-α2 IFN activity in the antiviral assay (summarized in Table 4A; FIG. 4). These include the charge-reversal mutants R120E, R120E/K121E and R120E-8CTail. In addition, although the 4-site alanine substitution mutant NLYY, mutated on helices B and C, retains about 1% of its antiviral activity (i.e., it is a weak agonist), the combination of L117A or R120A with NLYY leads to the loss of residual antiviral activity, and the gain of antagonist function (Table 4A).

The various mutants were also tested for their antagonism of antiproliferative activity (Table 4A), with results parallel to those obtained in the antiviral assays. As expected from the antiviral results, the R120E, R120E/K121E, NLYY-117A and NLYY-120A mutants are antagonists of IFN-α2. In addition, several mutants that preferentially lost antiproliferative activity while retaining low anti viral activity, such as R120A/K121A, L117A/R120A/K121A and Y85A/Y89A/R120A, had weak antiproliferative antagonist activity.

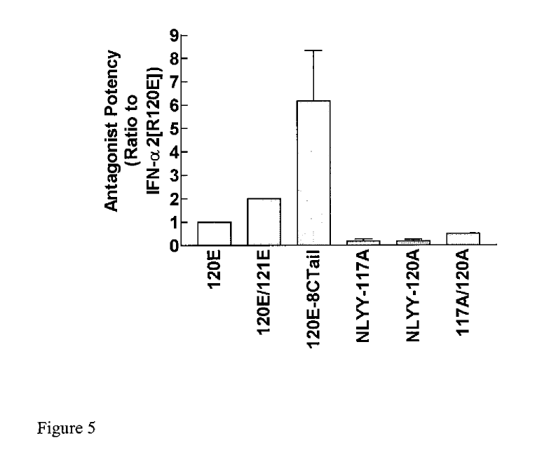

The antagonists vary in their potencies (FIG. 5; Table 4A). The R120E/K121E and R120E mutants are more potent than the NLYY-117A and NLYY-120A mutants that require higher concentrations for antagonism, in addition, several mutants are only weakly inhibitory. It is hypothesized that the greater potency derives from more complete disruption of the IFNAR-1 binding, although for all antagonists for which there was data available, the binding to IFNAR-1 has a K_{D} of ≧10 μM. For the antiviral assays, the molar ratio of R120E to native IFN for full antagonism is 100-250. Since studies have demonstrated that cellular activation for some assays requires only 5-10 percent of receptor occupancy, it is likely that effective blockade of this activity requires almost complete saturation of receptors; in the antiviral assay, even a small number of unblocked receptors left unblocked by the antagonist for a short period would permit binding of native IFN and development of viral resistance. For antiproliferative assays, the molar ratio of R120E or R120E/K121E to native IFN for effective antagonism is lower (range 16-100), probably reflecting the need for sustained active IFN action for achieving antiproliferative effects.

An effective in vivo antagonist will require higher potency through stronger binding to IFNAR-2. As a first step, a derivative of the IFN-α2[R120E] mutant was constructed that also had substitutions in the C-terminal tail such that the C-terminus had the more basic sequence of IFN-α8 (KRLKSKE), rather than that of IFN-α2 (ESLRSKE), which is denoted “120E-8CTail”. It was previously shown that IFN-α8 binds more strongly to IFNAR-2, and that replacement of 3 amino acids in the C-terminus of native IFN-α2 by those found in IFN-α8 increases the affinity of the IFN-α2 C-terminal mutant for IFNAR-2. Therefore, 120E-8CTail, with its higher affinity for IFNAR-2 was expected to have higher potency in the antagonism assay. This prediction was validated: 120E-8CTail can antagonize IFN-α2 at a concentration 4-to-8-fold lower than IFN-α2[R120E] (FIG. 5).

### Discussion

Recent evidence implicating Type I IFNs in the pathogenesis of systemic lupus erythematosus and possibly Sjögren's syndrome and other autoimmune diseases motivates the development of effective antagonists for Type I IFNs. The current strategy for a Type I IFN antagonist is to disable the IFNAR-1 site while maintaining or improving the affinity of the IFNAR-2 site. An analogous strategy has been employed in developing ligand-based antagonists for other cytokines that require ligand-dependent ligation of 2 receptor subunits to initiate signaling. However, it was first necessary to more completely map the IFNAR-1 site and to find appropriate mutants in this site. The current results document that appropriate mutants of the IFNAR-1 site can serve as competitive antagonists of in vitro activities of IFN-α2.

Most recent attempts to identify the IFNAR-1 site have focused on helices B and C and the connecting loop BC of IFN-α. The most complete mutagenesis study, which included affinity measurements with IFNAR-1 and IFNAR-2, demonstrated for IFN-α2 that Phe64, Tyr85 and Tyr89 of helices B and C form a patch that interacts directly with IFNAR-1 and plays a strong role in the biological activity in IFN-α2. This region is referred to as “Site 1A”. However, while the four-point Site 1A mutant (“NLYN”) of IFN-α2 showed significant loss of activity, it was still a weak agonist, retaining 1% antiviral activity and 0.1% antiproliferative activity in vitro (Table 1A, FIG. 5).

The current data demonstrate that residues of the D-helix are also part of the IFNAR-1 site, and contribute to biological activity for two distinct IFN-αs, IFN-α2 and IFN-α2/α1 (Tables 1A and 3A). Most dramatically, substitution of alanine at position 120 (Arg for IFN-α2, Lys for IFN-α2/α1) strongly decreases antiviral and antiproliferative activity on human cells and, for IFN-α2/α1, on murine cells. This is the first “hot-spot” residue reported for the interaction with IFNAR-1. Charge-reversal mutations at position 120 resulted in total or near-total loss of antiviral activity for both IFN-α2 and IFN-α2/α1. Significantly, the IFN-α2/α1[R120E] mutant lost activity on both human and murine cells (Table 3A), demonstrating the conservation of sequence and function between the human and murine models. The importance of the conserved positive charge at position 120 suggests that it may form a salt-bridge with a negative charge on human, murine and possibly bovine IFNAR-1. In addition, Leu117, conserved in all human and murine Type I IFNs, is also implicated in IFNAR-1 binding, particularly when L117A is combined with R120A. Therefore, the residues may be implicated in the following order of importance: R120>>L117, with a lesser role for K/R121.

Data on the contributions of the D-helix follow several prior studies that demonstrated a role for the D-helix in biological activity. These findings varied widely in the IFNs used, the residues mutated, and the assays used, and most documented relatively small decreases in biological activity following mutagenesis. An exception is the study of Cheetham et al., where a two-site charge-reversal mutant of human IFN-α4 in the equivalent positions of R120 and K121 produced dramatic decreases of antiviral activity on human and bovine cells. Since that study preceded the determination of the 3-dimensional structure of IFN or knowledge that the IFN receptor is heterodimeric, a specific structural interpretation could not be provided, as is now possible. It should be noted that at least one study also provided inferential data that residues of the D-helix might interact with IFNAR-1.

The relationship of the Helix L) residues to the previously identified Site 1A is unknown, since the structure of the IFNAR-1/IFN complex is not known. The simplest model would consider that the relevant residues on Helix D are an extension of Site 1A, forming an extended binding site on Type I IFN for binding to a single binding site on IFNAR-1. This view is consistent with the low-resolution structure of the IFN-α2/IFNAR-1-ECD/IFNAR-2-ECD complex as obtained by density-modeling to a 3-dimensional image reconstruction of negatively stained electron microscopy images. Indeed, in this model, R120 is perfectly located to interact with IFNAR-1, and is proximal to an aspartic acid on IFNAR-1, making this salt-bridge a testable hypothesis. A possible, but less likely, alternative is that the residues on Helix D may be part of a site proximal to Site 1A, but distinct from it, so that R120 interacts with a second site on IFNAR-1, physically separate from the site binding to Site 1A. Multi-site interactions between IFNAR-1 and IFNs could also be consistent with the low-resolution model of the ternary IFN:IFNAR-1:IFNAR-2 complex derived from electron microscropy, and might provide greater opportunities for discriminating among the Type I IFNs, and modulating cellular responses. This scenario is reminiscent of the IL-1 receptor (IL-1R) binding interaction where the 3 extracellular immunoglobulin-like domains of IL-1R wrap around the IL-1β and IL-1RA ligands. However, for the IFN:IFNAR-1 interaction, more structural data is needed.

It has thus been demonstrated that mutants of HuIFN-α2 with deficient binding to IFNAR-1, and no detectable antiviral or antiproliferative activity, can function as novel antagonists of native IFN-α2 in antiviral and antiproliferative assays. The results fit a simple biophysical model of binding to a heterodimeric receptor, where decreasing or eliminating binding to one receptor subunit, while maintaining or enhancing binding to the other receptor subunit, can be used to generate and optimize antagonist activity. The antiviral assay, in particular, is a stringent test of antagonism, since small amounts of native IFN, acting during a short incubation, are sufficient to trigger antiviral protection. Thus, any antagonist must be present at sufficient concentration and have sufficient affinity for IFNAR-2 to effectively block the receptor. These IFN antagonists also provide further pharmacological evidence that recruitment of IFNAR-1 is required for the activities measured.

These novel antagonists are useful for in vitro inhibition of IFN. The current limitation for in vitro or therapeutic use is their potency, which reflects their affinity for IFNAR-2. Derived from IFN-α2, the antagonists have a K_{D} for IFNAR-2 of 1-3 nM, requiring them to be in high molar excess of native IFNs, some of which have even higher affinity for IFNAR-2 than does IFN-α2. As a first step toward increasing potency, it was demonstrated that changes in the C-terminus of the IFN-α2[R120E] mutant to produce the 120E-8CTail mutant increased the potency of antagonism, as expected from its higher affinity for IFNAR-2 (Table 1A; FIG. 5).

Several of the mutated residues, such as the positively charged position 120, are conserved in IFNs from other species, and it has been demonstrated that the IFN-α2/α1[R120E] mutant lacked antiviral and antiproliferative activity on both human and murine cells (Table 3A). Thus, mutation of equivalent residues may provide competitive antagonists for these species.

Data reported here adds to the understanding of the IFNAR-1 binding site on Type I IFNs and forms the basis for developing, novel antagonistic Type I IFN analogues that may provide useful alternatives to the more common antibody-based and receptor-based antagonists. Building on the examples reported here should be possible to develop antagonists with higher affinity for IFNAR-2 that will have the potency required for in vivo and therapeutic use.

TABLE 1A
Biological activity and binding to human IFN
receptor subunits of IFN-α2 and its mutants

| | | | | Antiproliferative |
|---|---|---|---|---|
| | Antiviral Activity | | | Activity |
| | (% of Native EC₅₀) | | | (% of Native EC₅₀) |
| | Human | Human | Bovine | Human |
| | (Wish) | (HeLa) | (MDBK) | (Wish) |
| IFN-α2 | 100 | 100 | 100 | 100 |
| -114A | 130 | 37 | 79 | 98 |
| -117A | 14 | 20 | 36 | 18 |
| -120A | 1.4 | 2.3 | 68 | 0.0 [symbol] |
| -125A | 76 | 90 | 100 | 83 |
| -114R | 86 | 49 | 94 | 46 |
| -120E | ≦0.05 | <0.028 | 30 | ≦0.05 |
| -120E-8CTail | — | <0.028 | — | — |
| -125E | 113 | 167 | — | 41 |
| -114A120A | 1. [symbol] | 1.0 | 11.7 | 0.2 |
| -117A120A | 0. [symbol] | <0.008 | 0.7 | ≦0.05 |
| -120A121A | 3.1 | 1.6 | 1 [symbol] | ≦0.09 |
| -120E121E | ≦0.002 | <1.3 | 10 | ≦0.02 |
| -117A120A121A | 0.3 | ≦0.016 | 4. [symbol] | ≦0.05 |
| -NLYY | 1.3 | 1.8 | 39 | ≦0.1 |
| -NLYY-114A | 1.9 | 1.0 | 13 | 0.05 |
| -NLYY-117A | 0.03 | <0.03 | 13 | ≦0.05 |
| -NLYY-120A | ≦0.001 | ≦0.002 | — | ≦0.001 |
| -85A89A-120A | 0.5 | 0.012 | 0.72 | ≦0.03 |

The antiviral activity of IFN-α2 is 2-4 × 10⁸ units/mg on human HeLa cells challenged with VSV, calibrated against an international standard for IFN-α2. The native sequence contains the residues R120, K121, R125.
Binding affinity ratios are relative to wild type IFN-α2 affinities towards IFNAR1 (K_{D} = 2 μM) and IFNAR2 (K_{D} = 2 nM).
“ND”: “Not Detected”; binding below the detection limit of the measurement (K_{D} > 10 μM).
“—” not tested.
“NLYY” = N65A/L80A/Y85A/Y89A [symbol]
indicates data missing or illegible when filed

TABLE 2A
Antiviral and antiproliferative activities of IFN-α2/α1 and its Site 1A mutants

| | Antiviral Activity | | | | Antipro [symbol] |
|---|---|---|---|---|---|
| | (% of native) | | | | ( [symbol] |
| | Human | Human | Murine | Bovine | Human |
| | (Wish) | (HeLa) | (L-929) | (MDBK) | (Wish) |
| IFN-α2/α1 | 100 | 100 | 100 | 100 | 100 |
| -85A | — | 141 | 144 | 55 | — |
| -89A | 96 | 69 | 38 | 100 | 97 |
| -80A* | 19* | — | 31 | 34 | — |
| -85A89A | 183 | 143 | — | — | 89 |
| -80A85A89A | 204 | 54 | 100 | 100 | 22 |
| -65A80A85A89A | [symbol] | [symbol] | 55 | 200 | 16 |
| -65A80A [symbol] A89A-64A | 11 | 4.2 | — | — | 2.3 |

The antiviral activity of IFN-α2/α1 is 1-2 × 10⁸ units/mg on human HeLa cells challenged with VSV, calibrated against an international standard for IFN-α2. The native sequence of IFN-α2/α1 contains the residues: N65, L80, C85, Y89.
“—” not tested.
*Assay on human A549 cells. [symbol]
indicates data missing or illegible when filed

TABLE 3A
Biological activity and binding to human IFN receptor
subunits of IFN-α2/α1 and its mutants

| | | | | | Antiproliferative | |
|---|---|---|---|---|---|---|
| | Antiviral Activity | | | | Activity | |
| | (% of Native) | | | | (% of native) | |
| | Human | Human | Murine | Bovine | Human | Murine |
| | (Wish) | (HeLa) | (L-929) | (MDBK) | (WISH) | (NFS-01) |
| IFN-α2/α1 | 100 | 100 | 100 | 100 | 100 | 100 |
| -120A | 9.7 | 2 | 2 | 100 | 0.7 | 0.03 |
| -120A/121A | 13.8 | 8.3 | 33 | 100 | 0.5 | 8 |
| -120E | ≦0.02 | <0.04 | ≦0.04 | 100 | <0.02 | ≦0.0005 |
| -120E/121E | 0.006 | <0.08 | ≦0.04 | 10 | ≦0.05 | <0.0001 |
| -121E | — | 8 | 0.5 | 100 | — | — |
| -124E | — | 20 | 100 | 100 | — | — |
| -120E/121E/124E | <0.1 | <0.04 | <0.01 | <0.08 | <0.2 | — |

The antiviral activity of IFN-α2/α1 is 1-2 × 10⁸ units/mg on human HeLa cells challenged with VSV, calibrated against an international standard for IFN-α2. The native sequence contains the residues K120, K121, R124.
Binding affinity ratios are relative to wild type IFN-α2 affinities towards IFNAR1 (K_{D} = 2 μM) and IFNAR2 (K_{D} = 2 nM) and not relative to IFNα2/α1.
* The dissociation rate (k_{d}) of this mutant from IFNAR2 is similar to wild type IFN-α2. Change in the affinity stems from change in association rate (k_{d}), possibly due the additional charges presented by the added negatively charged residues that act over long distances.
“—” not tested.
“ND”: “Not Detected”; binding below the detection limit of the measurement (K_{D} > 10 μM).

TABLE 4A
Antagonist properties of human IFN-α2 variants.

| | | Anti- | | |
|---|---|---|---|---|
| | Antiviral | proliferative | Antagonist Activity³ | |
| | Activity¹ | Activity¹ | | Anti- |
| | Human | Human | Antiviral | proliferative |
| Interferon Form | (Wish; %) | (Wish; %) | IC₅₀ (M)⁴ | IC₅₀ (M)⁵ |
| IFN-α2 (wild-type) | 100 | 100 | No | No |
| -114A | 130 | 98 | No | No |
| -117A | 14 | 18 | No | No |
| -120A | 1.4 | 0.05 | No | +/− |
| -120E | ≦0.05 | ≦0.05 | 4.1 × 10⁻⁹ | 5.3 × 10⁻⁹ |
| -125E | 113 | 41 | No | No |
| -117A120A | 0.15 | ≦0.05 | No | YES |
| -120A121A | 3.1 | ≦0.09 | No | +/− |
| -120E121E | ≦0.002 | ≦0.02 | 2.1 × 10⁻⁹ | 2.6 × 10⁻⁹ |
| -117A120A121A | 0.3 | ≦0.05 | +/− | YES |
| -NLYY | 1.3 | ≦0.1 | No | No |
| -NLYY-117A | <0.03 | ≦0.05 | 4.1 × 10⁻⁸ | 8.5 × 10⁻⁸ |
| -NLYY-120A | ≦0.001 | ≦0.001 | 4.1 × 10⁻⁸ | 1.7 × 10⁻⁷ |
| -85A89A-120A | 0.5 | ≦0.03 | — | YES |

“NLYY” = N65A/L80A/Y85A/Y89A. The native sequence residues are: D114, L117, R120, K121, Q124.
¹Activity expressed as % of native EC₅₀. .
⁴Representative data, competing with 1.3 × 10⁻¹⁰ M IFN-α2 (HeLa cells).
⁵Representative data, competing against 1.7 × 10⁻⁹ M IFN-α2 (WISH cells).
“NO” corresponds to agonist activity.
“+/−”: variable/weak.
“—” not tested.
“YES” - antagonism was demonstrated, but quantitative variability between assays do not permit direct comparison to the IC₅₀ values reported for other mutants in this table.

TABLE 5A
Alignment of homologous amino acid sequences for human and mouse
Type 1 interferons

| Hu-alpha2 ---- | | |
|---|---|---|
| CDLPQTHSLGSRRTLNLLAQMRKISLFSCLKDRHDFGFPQEEF--CHQFQKAETIP | | 54 |
| Hu-alpha1 ---- | | |
| CDLPETHSLDHRRTLMLLAQMSRISPSSCLMDRHDFGFPQEEP-DGNQFQKAPAIS | | 55 |
| Hu-alpha2/1 ---- | | |
| CDLPQTHSLGSRRTLNLLAQMRKISLFSCLKDRHDFGFPQEEF--GNQFQKAETIP | | 54 |
| Mu-alpha4 --- | | |
| CDLPHTYLNGNKRALTVLEEMRRKPPLSCKLDRKDFGFPLEKVD-NQQIQKAQAIL | | 55 |
| Mu-alpha12 ---- | | |
| CDLPQTHNLRNKRALTLLAQMRRLSPLSCLKDRKNFRFPQEKVD-AQQIKKAQVIP | | 55 |
| Hu-omega ---- | | |
| CDLPQNHGLLSRNTLVLLHQMRRISPFLCLKDRRDPRFPQEMVK-GSQLQKAHVNS | | 55 |
| Hu-beta --- | | |
| SYNLLGFLQRSSNCQCQKLLWQLN-GRLEYCLKDRRNFDIPEEIKQ-LQQFQKEDAAV | | 56 |
| Hu-kappa | | |
| SLDCNLLNVHLRRVTWQNLRHLSSMSNSFPVECLRENIAFELPQEFLQ-YTQPMKRDIKK | | 59 |
| Hu-epsilon | | |
| SLDLKLYIFQQRQVNQESLKLLNKLQTLSIQQCLPHRKNFLLPQKSLS-PQQYQKGHTLA | | 59 |
| Mu-beta -- | | |
| INYKQLQLQERTNYRKCQELLEQLN-GKIN---LTYRADFKIPMENT---EKNQKSYTAF | | 53 |
| Mu-limitin - | | |
| SLDSGKSGSLHLERSETARFLAELRSVPGHQCLRDRTDFPCPWKEGTNITQMTLGETTS | | 59 |
| Hu-alpha2 | | |
| VLHEMIQQIFNLFSTKDSSAAWDETLLDKFYTELYQQLNDLEACVIQGVGVTETPLMKED | | 114 |
| Hu-alpha1 | | |
| VLHELIQQIPNLFTTKDSSAAWDEDLLDKFCTELYQQLNDLEACVMQEERVGETPLMNAD | | 115 |
| Hu-alpha2/1 | | |
| VLHEMIQQIPNLFTFKDSSAAWDEDLLDKFCTELYQQLNDLEACVMQEERVGETPLNNAD | | 114 |
| Mu-alpha4 | | |
| VLRDLTQQILNLFTSKDLSATWNATLLDSFCNDLHQQLNDLKACVMQ-----EPPLTQED | | 110 |
| Mu-alpha12 | | |
| VLSELTQQILTLFTSKDSSAAWNTTLLDSFCNDLHQQLNDLQGCLMQQVGVQEPPLTQED | | 115 |
| Hu-omega | | |
| VLHEMLQQIPSLFHTERSSAAWNMTLLDQLHTGLHQQLQHLETCLLQVVGEGESAGAISS | | 115 |
| Hu-beta | | |
| TIYEMLQHIFAIFRQDSSSTGWNETIVENLLANVYHQRNHLKTVLEEKLEKEDFTRGKRM | | 116 |
| Hu-kappa | | |
| AFYEMSLQAPNIFSQH-TFKYWKERHLKQIQIGLSQQAEYLNQCLEEDENENEDWKEMKE | | 118 |
| Hu-epsilon | | |
| ILHEMLQQIFSLPRANYSLDGWEENHTEKFLIQLHQQLEYLEALMGLEAEKLSGTLGSDN | | 119 |
| Mu-beta | | |
| AIQEMLQNVFLVFRNNPSSTGWNETIVVRLLDELHQQTVFLKTVLEEKQE-ERLTWEMSS | | 112 |
| Mu-limitin | | |
| CYSQTLRQVLHLFDTEASRAAWHERALDQLLSSLWRELQVLKRPREQGQSCPLPFA---- | | 115 |
| Hu-alpha2 ------------- | | |
| SILAVRKYPQRITLYLKEKKYSPCAWEVVRAEIMRSFSLSTNLQESL | | 161 |
| Hu-alpha1 ------------- | | |
| SILAVKKYFRRITLYLTEKKYSPCAWEVVRAEIMRSLSLSTNLQERL | | 162 |
| Hu-alpha2/1 ------------- | | |
| SILAVKKYFRRITLYLTEKKYSPCAWEVVRAEIMRSLSLSTNLQERL | | 161 |
| Mu-alpha4 ------------- | | |
| SLLAVRTYFHRITVYLRKKKHSLCAWEVIRAEVWRALSSSTNLLARL | | 157 |
| Mu-alpha12 ------------- | | |
| SLLAVRKYFHRITVYLREKKHSPCAWEVVRAEVWRTLSSSAKLLARL | | 162 |
| Hu-omega ------------- | | |
| PALTLRRYFQGIRVYLKEKKYSDCAWEVVRMEIMKSLPLSTNNQERL | | 162 |
| Hu-beta ------------- | | |
| SSLHLKRYYGRILHYLKAKEDSHCAWTIVRVEILRNFYVINRLTGYL | | 163 |
| Hu-kappa | | |
| NEMKPSEARVPQLSSLELRRYFHRIDNFLKEKKYSDCAWEIVRVEIRRCLYYFYKPTALF | | 178 |
| Hu-epsilon ------------- | | |
| LRLQVKMYFRRIHDYLENQDYSTCAWAIVQVEISRCLFFVFSLTEKL | | 166 |
| Mu-beta ------------- | | |
| TALHLKSYYWRVQRYLKLMKYNSYAWNVVRAEIFRNPLIIRRLTRNF | | 159 |
| Mu-limitin --------------- | | |
| LAIRTYPRGFFRYLKAKAMSACSWEIVRVQLQVDLPAFPLSARRG | | 160 |
| Hu-alpha2 | RS-KE----------------- | 165 |
| Hu-alpha1 | RR-KE----------------- | 166 |
| Hu-alpha2/1 | RR-KE----------------- | 165 |
| Mu-alpha4 | SEEKE----------------- | 162 |
| Mu-alpha12 | SE-KE----------------- | 166 |
| Hu-omega | RS-KDRDLGSS----------- | 172 |
| Hu-beta | RN-------------------- | 165 |
| Hu-kappa | RRK------------------- | 181 |
| Hu-epsilon | SKQGRPLNDNKQELTTKFRSFR | 188 |
| Mu-beta | QN-------------------- | 161 |
| Mu-limitin | FR-------------------- | 162 |

Table 5A. Alignment of homologous amino acid sequences for some diverse human and mouse Type 1 interferons, according to the predicted alignment generated by one computer program commonly used to predict equivalent positions or regions of related proteins.
Letters refer to the single-letter code for amino acids.
Dashes(-) refer to positions not found in an amino acid sequence but which have an amino acid at an equivalent position of another interferon.
The numbers correspond to the amino acid position numbers of the specific protein sequence, where the number “1” for each sequence is the first amino acid expected for the secreted (“mature”) version of each protein, and varies slightly among the interferon.
The prefix “hu” refers to human interferons, “mu” refers to mouse (murine) proteins.

We claim:

1. A method of preparing a Type I interferon antagonist comprising modifying a Type 1 interferon at the site of interaction with the interferon receptor subunit IFNAR-1 such that the binding affinity of the interferon to the IFNAR-1 subunit is reduced as compared to the native interferon.

2. The method of claim 1, wherein the binding affinity of the interferon to the IFNAR-2 subunit is maintained as compared to the native interferon.

3. The method of claim 1, further comprising modifying the interferon at the site of interaction with the interferon receptor subunit IFNAR-2 such that the binding affinity of the interferon to the IFNAR-2 subunit is increased as compared to the native interferon.

4. A method of preparing a Type I interferon antagonist comprising modifying an interferon such that (i) the binding affinity of the interferon to the IFNAR-1 subunit is reduced as compared to the native interferon and (ii) the binding affinity of the interferon to the IFNAR-2 subunit is increased as compared to the native interferon.

5. The method of any claims 1-4, wherein the interferon is selected from the group consisting of IFN-α, IFN-β, IFN-ω, IFN-κ, INF-ε, IFN-τ, IFN-ζ/limitin, IFN-δ and IFN-ν.

6. The method of claim 1, wherein the modifying comprises mutating, one or more amino acids in the IFNAR-1 binding region of the interferon.

7. The method of any of claims 1-4, wherein the interferon originates from a mammal.

8. The method of claim 8, wherein the mammal is a human or a mouse.

9. The method of any of claims 1-4, wherein the interferon is IFN-α2, preferably IFN-α2a or IFN-α2b.

10. The method of claim 9, wherein the IFN-α2b is modified at one or more amino acid positions in region 120-125.

11. The method of claim 10, wherein the IFN-α2b is modified at one or more sites selected from the group consisting of Arg120, Lys121 and Gln124.

12. The method of claim 11, wherein the Arg 120 of the IFN-α2b is substituted with Glu.

13. The method of claim 11, wherein the Arg 120 of the IFN-α2b is substituted with Glu and the Lys 121 is substituted with Glu.

14. A Type I interferon produced according to any of the methods of claims 1-13.

15. A Type I interferon that has sufficiently low binding affinity to the interferon receptor subunit IFNAR-1 such that the interferon exhibits antagonist activity.

16. The Type I interferon of claim 15 that has a sufficient binding affinity to the interferon receptor subunit. IFNA R-2 to interfere with the binding of a native or endogenous interferon.

17. A Type I interferon that has (i) sufficiently low binding affinity to the interferon receptor subunit IFNAR-1 such that the interferon exhibits antagonist activity and (ii) sufficient binding affinity to the interferon receptor subunit IFNAR-2 to interfere with the binding of a native or endogenous interferon

18. The Type I interferon of any of claims 15-17, wherein the interferon is selected from the group consisting of IFN-α. IFN-β, IFN-ω, IFN-κ, IFN-ε, IFN-τ, and IFN-ζ/limitin, IFN-δ and IFN-ν.

19. The Type I interferon of any of claims 15-17, wherein the interferon originates from a mammal.

20. The Type I interferon of claim 20, wherein the mammal is a human or a mouse.

21. The Type I interferon of any of claims 15-17 wherein the interferon is IFN-α2b.

22. The Type I interferon of any of claims 15-17, having a Glu at the 120 amino acid position.

23. The Type I interferon of any of claims 15-17, having a Glu at the 121 amino acid position.

24. The Type I interferon of any of claim 15-17, having a Glu at the 120 and 121 amino acid position.

25. A method of antagonizing the effects of interferon comprising contacting an interferon receptor with a Type I interferon antagonist of any of claims 14-17.

26. The method of claim 26, wherein the contacting is in-vitro or in-vivo.

27. A method of treating a disease or condition in a mammal comprising administering a Type I interferon antagonist of any of claims 14-17, in an effective amount to antagonize the effects of a native or endogenous interferon.

28. The method of claim 27, wherein the disease or condition is auto-immune mediated.

29. The method of claim 27, wherein the disease or condition is selected from the group consisting of systemic lupus erythematosus, Sjogren's syndrome, Type 1 diabetes, polymyositis, and periodontitis.

30. The method of claim 27, wherein the administration is associated with allogeneic grafts or transplants.

31. A method of treating a disease or condition in a mammal comprising administering a nucleic acid encoding a Type I interferon antagonist of any of claims 14-17, in an effective amount to antagonize the effects of a native or endogenous interferon.

32. The method of claim 31, wherein the nucleic acid comprises DNA.

33. The method of claim 31, wherein the nucleic acid comprises RNA.

34. The method of claim 31, wherein the nucleic acid is contained within a vector.

35. The method of claim 34, wherein the vector is a plasmid.

36. The method of claim 34, wherein the vector is a virus.

37. A nucleic acid encoding a Type I interferon antagonist of any of claims 14-17.

38. The nucleic acid of claim 37, wherein the nucleic acid comprises DNA.

39. The nucleic acid of claim 37, wherein the nucleic acid comprises RNA.

40. The method of claim 31, wherein the disease or condition is auto-immune mediated.

41. The method of claim 31, wherein the disease or condition is selected from the group consisting of systemic lupus erythematosus, Sjogren's syndrome; Type 1 diabetes, polymyositis, and periodontitis.

42. The method of claim 31, wherein the administration is associated with allogeneic grafts or transplants.

43. The method of any of claims 27-36, wherein the administration is selected from the group consisting of parenteral, intradermal, intramuscular, intraperitoneal, intravenous, subcutaneous, intranasal, epidural, topical, pulmonary and oral routes.

44. A pharmaceutical composition comprising the interferon of any of claims 14-24 and a pharmaceutically acceptable excipient.

45. The pharmaceutical composition of claim 44, in a form selected from the group consisting of a solution, suspension, emulsion, tablet, capsule, powder and sustained-release formulation.
